(19) United States
(12) Patent Application Publication
(10) Pub. No.: US 2005/0112783 A1
(43) Pub. Date: May 26, 2005

(54) Non-pressure based fluid transfer in assay detection systems and related methods

(75) Inventors: Douglas Evans, San Diego, CA (US); Nick Callamaras, San Diego, CA (US)

Correspondence Address: QUINE INTELLECTUAL PROPERTY LAW GROUP, P.C., P O BOX 458, ALAMEDA, CA, 94501, US

(73) Assignee: IRM, LLC, Hamilton (BM)

(21) Appl. No.: 10/911,388

(22) Filed: Aug. 3, 2004

Related U.S. Application Data
(60) Provisional application No. 60/492,629, filed on Aug. 4, 2003.

(51) Int. Cl. C12M 1/34; G01N 33/543; C12Q 1/68

(52) U.S. Cl. 436/518; 435/287.2; 435/6

(57) ABSTRACT

The present invention relates generally to sample assaying systems that include non-pressure-based fluid transfer probes. The non-pressure-based fluid transfer probes of the invention are typically utilized to transfer fluidic samples to sample assay containers or other supports for sample detection or imaging. The invention also provides various additional components that are optionally included in these systems, including container positioning devices, container storage components, and robotic devices among others. Pin tools and methods of assaying fluidic samples that utilize the systems of the invention are also provided.

### CROSS-REFERENCES TO RELATED APPLICATIONS

This application claims the benefit of U.S. Provisional Application No. 60/492,629, filed Aug. 4, 2003, the disclosure of which is incorporated by reference in its entirety for all purposes.

### COPYRIGHT NOTIFICATION

Pursuant to 37 C.F.R. § 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates generally to fluid transfer and assay detection, and more particularly, to methods, systems, and system components for non-pressure-based fluid transfer and detection of electromagnetic radiation in samples.

### BACKGROUND OF THE INVENTION

High-throughput screening devices and systems are important analytical tools in the pharmaceutical research industry and in the process of discovering and developing new drugs. Drug discovery procedures typically involve synthesis and screening of candidate drug compounds against selected targets. Candidate drug compounds are molecules with the potential to modulate diseases by affecting given targets. Targets are typically biological molecules, including proteins such as enzymes and receptors, or nucleic acids, that are thought to play roles in the onset or progression of particular diseases. A target is typically identified based on its anticipated role in the progression or prevention of a disease. Recent developments in molecular biology and genomics have led to a dramatic increase in the number of targets available for drug discovery research.

Once a target is selected, a library of compounds is typically selected to screen against the target. Enormous compound libraries have been compiled from natural sources and via various synthetic routes, including combinatorial synthesis schemes. In fact, many pharmaceutical companies and other institutions have access to libraries that include hundreds of thousands of compounds. Following the selection of a target and compound library, the compounds are screened to determine if they have any affect on the target. Compounds that affect the target are denominated as hits. A basic premise for screening larger numbers of compounds against a particular target is the increased statistical probability of identifying a hit.

Before screening compounds against a target, the assay is developed. The assay development process includes selecting and optimizing an assay that will measure the performance of a compound against the selected target. Assays are generally classified as either biochemical or cellular. Biochemical assays are typically performed with purified molecular targets, whereas cellular assays are performed with living cells. While cellular assays often provide more biologically relevant information than biochemical assays, they are typically more complex and time-consuming to perform than biochemical counterparts.

In performing biochemical and cellular assays, samples are routinely characterized by examining properties, such as fluorescence, luminescence, and absorption. In a fluorescence study, for example, selected tissues, specific binding partners, chromosomes, or other structures are treated with a fluorescent probe or dye. The sample is then irradiated with light of a wavelength that causes the fluorescent material to emit light at a longer wavelength, thus allowing the treated structures to be identified and to at least some extent quantified. In a luminescence analysis, the sample is not irradiated in order to initiate light emission by the material. Instead, one or more reagents are typically added to the sample in order to initiate the luminescence phenomena. In an absorption analysis, a dye-containing sample is typically irradiated by an electromagnetic radiation source of a selected wavelength. The amount of light transmitted through the sample is generally measured relative to the amount of light transmitted through a reference sample without dye. Analytical devices and systems utilized to determine the fluorescence of a sample typically include at least one electromagnetic radiation source capable of emitting radiation at one or more excitation wavelengths and a detector for monitoring the fluorescence emissions. In many cases, these devices and systems can also be adapted for use in both luminescence and absorption analyses.

To accommodate the large number of compounds and targets, multiple screens are often performed in parallel in the wells of standard multi-well containers of selected well-densities and even on the surfaces of various supports, such as membranes or treated glass. Parallel screens typically include transferring multiple samples from the wells of compound plates to the wells of a corresponding assay plate or the surface of a support, e.g., prior to the detection of fluorescent, luminescent, and/or absorption properties of the samples. Many conventional systems are limited to transferring fluids using pressure-based fluid transfer devices, such as pipetting devices. While suitable for many applications, the cost of replacing pipette tips adds to the overall cost of performing compound screening. The cost of replacing these consumables can be substantial, given the large numbers of screens that are typically performed to identify hits. In addition, pipette tip openings can become obstructed by precipitate, cells, or other debris, which typically necessitates halting the screen in order to clear the obstruction or to replace the tip. This can severely limit the throughput of screening procedures, which have become increasing automated.

From the foregoing, it is apparent that assaying systems that at least include the option of using fluid transfer techniques that do not suffer from the limitations of pressure-based methods are desirable. These and a variety of additional features of the present invention will become evident upon complete review of the following.

### SUMMARY OF THE INVENTION

The present invention relates generally to sample assaying systems that include non-pressure-based fluid transfer probes. In particular, the non-pressure-based fluid transfer probes of the invention are typically utilized to transfer fluidic samples to sample assay containers or other supports for sample detection or imaging. In preferred embodiments, non-pressure-based fluid transfer probes of the systems described herein comprise pin tools having multiple pins for transferring multiple samples with greater throughput than fluid transfers involving pressure-based transfer devices, such as pipetting devices, the tips of which can become obstructed, thereby limiting assay throughput. The invention further provides various additional components that are optionally included in the systems of the invention, including container positioning devices, container storage components, and robotic devices among others. Pin tools and methods of assaying fluidic samples that utilize the systems of the invention are also provided.

In one aspect, the present invention provides a sample assaying system. The system includes at least one electromagnetic radiation source (e.g., a laser source, etc.), and at least one sample assaying region configured to receive source electromagnetic radiation from the electromagnetic radiation source. In some embodiments, the sample assaying region includes at least one thermal modulation nest, e.g., which modulates temperature in containers disposed on or proximal to the thermal modulation nest. The system also includes at least one fluid transfer device including at least one non-pressure-based fluid transfer probe (e.g., a pin or the like), which fluid transfer device is configured to transfer fluid in at least one selected region (e.g., the sample assaying region, etc.) of the sample assaying system. In preferred embodiments, the non-pressure-based fluid transfer probe is removably attached to the fluid transfer device. In addition, the system also includes at least one detector (e.g., a CCD camera or the like) configured to detect sample electromagnetic radiation received from the sample assaying region. In some embodiments, the sample assaying system further includes at least one controller operably connected at least to the electromagnetic radiation source, the fluid transfer device (e.g., a computer or the like), and the detector. The controller typically includes at least one logic device having one or more logic instructions that direct operation of the electromagnetic radiation source, the fluid transfer device, the detector, and optionally, other components of the system.

The non-pressure-based fluid transfer probe generally includes a pin tool that comprises, e.g., 6, 12, 24, 48, 96, 192, 384, 768, 1536, 3456, 9600, or more pins. In addition, the fluid transfer device typically comprises at least one chassis and the pin tool optionally comprises a support structure having at least one attachment feature that removably attaches to the chassis. In these embodiments, the system generally further includes at least one controller operably connected to the fluid transfer device. The controller generally includes at least one logic device having one or more logic instructions that direct the fluid transfer device to attach and/or detach the pin tool to or from the chassis, and the fluid transfer device to transfer fluid between containers with the pin tool. The attachment feature typically includes a hook or a functionally equivalent feature. Optionally, the pin tool includes a pin tool head having a rotational adjustment feature (e.g., a screw, etc.) such that the pin tool head is capable of rotating relative to the support structure, e.g., to adjust the alignment of the pin tool head with the sample container. The pin tool head is generally removably attached to the support structure by one or more attachment components, such as set screws, spring ball sockets, and/or the like.

The sample assaying region typically includes at least one container positioning device, which container positioning device comprises at least one container station that is structured to position at least one container or other support relative to the fluid transfer device. In certain embodiments, the container station includes a heating element to regulate temperature in the container or on the other support. The container station is typically structured to position at least one multi-well container that comprises, e.g., 6, 12, 24, 48, 96, 192, 384, 768, 1536, 3456, 9600, or more wells, e.g., configured to receive the pins of the pin tool. In some embodiments, the container station comprises a nest. Multi-well plates with high well-densities are generally preferred (e.g., plates having 1536 or more wells). Optionally, the container station is structured to rotate relative to the fluid transfer device, e.g., to align a container positioned in the station with the fluid transfer device. The container station generally comprises at least one orifice disposed through the container positioning device such that when one or more containers are positioned in the container station, the containers receive the source electromagnetic radiation from the electromagnetic radiation source through the orifice and/or the detector receives the sample electromagnetic radiation from the containers through the orifice.

In preferred embodiments, the container positioning device comprises multiple container stations. Typically, at least two of the container stations are tiered. In these embodiments, for example, the system generally further includes at least one robotic handler capable of handling a first container positioned in one tiered container station without contacting a second container positioned in another tiered container station.

In some embodiments, the container positioning device comprises one or more alignment members that are positioned to contact one or more surfaces of one or more containers when the containers are positioned in the container station such that the containers align with the fluid transfer device. For example, alignment member receiving areas disposed in bottom surfaces of the containers optionally include the surfaces that contact the alignment members. In these embodiments, the system typically further includes one or more pushers that are capable of pushing the containers into contact with the alignment members when the containers are positioned in the container station. In these embodiments, the system typically further includes at least one controller operably connected to the container positioning device. The controller typically includes at least one logic device having one or more logic instructions that direct the pushers to push the containers into contact with the alignment members when the containers are positioned in the container station.

In preferred embodiments, the sample assaying system further includes at least one fluid transfer probe washing station that comprises at least one wash reservoir structured to wash the non-pressure-based fluid transfer probe. The wash reservoir typically includes at least one overflow reservoir in fluid communication with the wash reservoir to receive fluid overflow from the wash reservoir. In some embodiments, the wash reservoir comprises at least one mount to position the non-pressure-based fluid transfer probe relative to the wash reservoir when the non-pressure-based fluid transfer probe is washed and/or when the non-pressure-based fluid transfer probe is separated from a chassis of the fluid transfer device. The fluid transfer probe washing station optionally includes at least a first alignment feature and the non-pressure-based fluid transfer probe comprises at least a second alignment feature, which alignment features are capable of mating with one another to align the non-pressure-based fluid transfer probe relative to the fluid transfer probe washing station. In certain embodiments, the system further includes at least one controller operably connected to the fluid transfer probe washing station. The controller typically directs operation of the fluid transfer probe washing station. In some embodiments, the system further includes at least one fluid sensor in sensory communication with at least one component of the fluid transfer probe washing station to sense fluid disposed proximal to the component. The fluid sensor is also optionally operably connected to the controller.

The fluid transfer probe washing station typically further includes at least one waste reservoir in fluid communication with the wash reservoir by at least one fluid conduit. In these embodiments, the system typically further comprises at least one pump operably connected to the fluid conduit to effect fluid flow through the conduit. In addition, the system also typically further includes at least one valve operably connected to the fluid conduit to regulate fluid flow through the fluid conduit.

The sample assaying systems of the invention optionally include various additional components. In some embodiments, for example, the system further includes at least one container storage component that is structured to store one or more containers and/or at least one container incubation component that is structured to incubate one or more containers. In certain embodiments, the system further includes a container moving component that is structured to move one or more containers at least relative to the fluid transfer device. In these embodiments, the system typically further includes at least one controller operably connected to the container moving component. The controller generally includes at least one logic device having one or more logic instructions that direct movement of the container moving component. In some embodiments, the system further includes at least one robotic device configured to translocate containers at least between selected regions of the sample assaying system. In these embodiments, the system typically further includes at least one controller operably connected to the robotic device. The controller typically includes at least one logic device having one or more logic instructions that direct the robotic device to translocate the containers.

In some embodiments of the invention, the system further includes at least one fluid transfer probe blotting station structured to blot away fluid that adheres to the non-pressure-based fluid transfer probe. In these embodiments, the system typically further includes at least one controller operably connected to the fluid transfer device. The controller generally includes at least one logic device having one or more logic instructions that direct the fluid transfer device to blot away the fluid that adheres to the non-pressure-based fluid transfer probe. In certain embodiments, the system further comprising at least one fluid transfer probe vacuum drying station structured to dry the non-pressure-based fluid transfer probe. In these embodiments, the system optionally further includes at least one controller operably connected to the fluid transfer device and the fluid transfer probe vacuum drying station. The controller typically includes at least one logic device having one or more logic instructions that direct the fluid transfer device to move the fluid transfer probe proximal to the fluid transfer probe vacuum drying station and the fluid transfer probe vacuum drying station to dry the fluid transfer probe.

In another aspect, the present invention relates to a sample assaying system. The system includes at least one electromagnetic radiation source and at least one container positioning device comprising at least one container station that is structured to position at least one multi-well container. The container station comprises at least one orifice disposed through the container positioning device such that when one or more multi-well containers are positioned in the container station at least one selected well disposed in the multi-well containers receives source electromagnetic radiation from the electromagnetic radiation source through the orifice. The system also includes at least one fluid transfer device comprising at least one chassis and at least one pin tool that removably attaches to the chassis. The pin tool is structured to transfer fluid to and/or from selected wells disposed in the multi-well container when the multi-well container is positioned in the container station. In addition, the system further includes at least one image detector configured to detect sample electromagnetic radiation received from the selected well disposed in the multi-well container through the orifice when the multi-well container is positioned in the container station.

In still another aspect, the invention provides a pin tool that includes a pin tool head having at least one pin attached to the pin tool head (e.g., floating, resiliently coupled to the pin tool head, etc.) and a support structure having at least one attachment feature (e.g., a hook, etc.) capable of removably attaching to a chassis of a fluid transfer device. The pin tool head is removably attached to the support structure and the pin tool head comprises a rotational adjustment feature (e.g., a screw or the like) such that the pin tool head is capable of rotating relative to the support structure. The pin tool head typically comprises, e.g., 6, 12, 24, 48, 96, 192, 384, 768, 1536, 3456, 9600, or more pins. In addition, the pin tool head is generally removably attached to the support structure by one or more attachment components, such as set screws, spring ball sockets, and/or the like.

In yet another aspect, the invention relates to a method of assaying fluidic samples. The method includes providing an assaying system that comprises at least one electromagnetic radiation source (e.g., a laser source or the like) and at least one sample assaying region configured to receive source electromagnetic radiation from the electromagnetic radiation source. The assaying system also includes at least one fluid transfer device comprising at least one non-pressure-based fluid transfer probe. The fluid transfer device is configured to transfer fluid in at least one selected region of the sample assaying system. In addition, the assaying system also includes at least one detector (e.g., a CCD camera or other detection device). The method also includes positioning at least a first container in the sample assaying region, and transferring at least one fluidic sample from at least a second container to the first container with the non-pressure-based fluid transfer probe. The method also includes detecting sample electromagnetic radiation received from the first container with the detector when the first container receives source electromagnetic radiation from the electromagnetic radiation source. The positioning step generally comprises placing the first container in the sample assaying region with a robotic device. In preferred embodiments, the detecting step comprises imaging the sample electromagnetic radiation received from the first container over time.

The transferring step typically comprises transferring multiple fluidic samples from the second container and/or at least a third container to the first container. Optionally, the method also includes washing, blotting, and/or drying the non-pressure-based fluidic transfer probe after at least one of the fluidic samples is transferred to the first fluid container. The non-pressure-based fluidic transfer probe typically comprises a pin tool having multiple pins and the transferring step comprises substantially simultaneously transferring multiple samples from the second container to the first container. In these embodiments, the fluid transfer device generally comprises at least one chassis and the pin tool comprises a support structure having at least one attachment feature that removably attaches to the chassis, and the method typically further comprises attaching the pin tool to the chassis before the transferring step or detaching the pin tool from the chassis after the transferring step. Optionally, the method further comprises rotating the pin tool and/or the first container relative to one another before or after transferring the multiple samples to the first sample container.

### BRIEF DESCRIPTION OF THE DRAWINGS

FIG. 1 schematically shows a sample assaying system from a perspective view according to one embodiment of the invention.

FIG. 2A schematically depicts a pin tool from a perspective view according to one embodiment of the invention.

FIG. 2B schematically illustrates the pin tool from FIG. 2A from another perspective view.

FIG. 2C schematically shows the pin tool from FIG. 2A from an exploded perspective view.

FIG. 2D schematically illustrates a pin tool support structure and a top plate of a pin tool head from an exploded perspective view according to one embodiment of the invention.

FIG. 2E schematically shows a pin tool from a perspective view according to one embodiment of the invention.

FIG. 2F schematically depicts the pin tool from FIG. 2E from an exploded perspective view.

FIG. 2G schematically illustrates the pin tool from FIG. 2E from an exploded front view.

FIG. 2H schematically shows an interface between components of a pin tool head from the pin tool of FIG. 2E from a detailed front view.

FIG. 3A schematically shows a chassis of a fluid transfer device from a perspective view according to one embodiment of the invention.

FIG. 3B schematically depicts a pin tool attached to the chassis of FIG. 3A.

FIG. 4 schematically shows a sample assaying region from a perspective view according to one embodiment of the invention.

FIG. 5 schematically depicts a support structure of a container positioning device from a bottom view according to one embodiment of the invention.

FIG. 6A schematically shows a front foot of a container positioning device from a detailed bottom view according to one embodiment of the invention.

FIG. 6B schematically illustrates the front foot of FIG. 6A from a detailed side view.

FIG. 6C schematically illustrates the front foot of FIG. 6A from a detailed top view.

FIG. 7A schematically shows a rear foot of a container positioning device from a detailed top view according to one embodiment of the invention.

FIG. 7B schematically illustrates the rear foot of FIG. 7A from a detailed side view.

FIG. 7C schematically illustrates the rear foot of FIG. 7A from a detailed bottom view.

FIG. 8A schematically shows a side view of the support structure shown in FIG. 5.

FIG. 8B schematically illustrates a cross-sectional side view of the support structure shown in FIG. 5.

FIG. 9A schematically shows the support structure shown in FIG. 5 from a top view.

FIG. 9B schematically depicts a cross-sectional side view of the support structure shown in FIG. 9A.

FIG. 9C schematically shows another cross-sectional side view of the support structure illustrated in FIG. 9A.

FIG. 9D schematically illustrates the support structure shown in FIG. 9A from a top perspective view.

FIG. 10A schematically shows a container positioning device that includes the support structure of FIG. 5 from a top view.

FIG. 10B schematically illustrates the container positioning device of FIG. 10A from a side elevational view.

FIG. 10C schematically illustrates the container positioning device of FIG. 10A from another side elevational view.

FIG. 10D schematically illustrates the container positioning device of FIG. 10A from a perspective view.

FIG. 10E schematically shows a perspective view of the positioning device of FIG. 10A mounted on a translational mechanism.

FIG. 10F schematically illustrates a sample assaying region from a perspective view according to one embodiment of the invention.

FIG. 10G schematically depicts a thermal modulation nest from a perspective view according to one embodiment of the invention.

FIG. 10H schematically shows the thermal modulation nest from FIG. 10G from a transparent top view.

FIG. 10I schematically shows a bottom plate of the thermal modulation nest from FIG. 10G from a top view.

FIG. 10J schematically illustrates the thermal modulation nest from FIG. 10G from a front view.

FIG. 10K schematically depicts the thermal modulation nest from FIG. 10G from a bottom view.

FIG. 11A schematically shows an alignment member of a container positioning device from a detailed top view.

FIG. 11B schematically depicts the alignment member of FIG. 11A from a detailed side view.

FIG. 11C schematically shows the alignment member of FIG. 11A from a detailed bottom view.

FIG. 12A schematically shows an alignment member of a container positioning device from a detailed top view.

FIG. 12B schematically depicts the alignment member of FIG. 12A from a detailed side view.

FIG. 12C schematically shows the alignment member of FIG. 12A from a detailed bottom view.

FIG. 13A schematically shows a pusher component from a detailed front view.

FIG. 13B schematically shows the pusher component of FIG. 13A from a detailed side view.

FIG. 13C schematically shows the pusher component of FIG. 13A from a detailed rear view.

FIG. 14A schematically shows a lever arm of a pusher from a detailed front view.

FIG. 14B schematically depicts the lever arm of FIG. 14A from a detailed rear view.

FIG. 14C schematically shows the lever arm of FIG. 14A from a detailed perspective view.

FIG. 15A schematically depicts a lever shaft of a pusher from a detailed front view.

FIG. 15B schematically illustrates the lever shaft of FIG. 15A from a detailed side view.

FIG. 15C schematically illustrates the lever shaft of FIG. 15A from a detailed top view.

FIG. 15D schematically shows the lever shaft of FIG. 15A from a detailed perspective view.

FIG. 16A schematically depicts a pin capture block of a pusher from a detailed top view.

FIG. 16B schematically shows the pin capture block of FIG. 16A from a detailed side view.

FIG. 16C schematically depicts the pin capture block of FIG. 16A from a detailed bottom view.

FIG. 17A schematically shows a container positioning device from a perspective view according to one embodiment of the invention.

FIG. 17B schematically shows the container positioning device of FIG. 17A from a partially exploded perspective view.

FIG. 17C schematically illustrates a partially transparent top view of a portion of a nest from the container positioning device of FIG. 17A.

FIG. 17D schematically shows the nest of FIG. 17C from a bottom perspective view.

FIG. 17E schematically depicts a detailed perspective view of the rotational coupling components shown in FIG. 17D.

FIG. 18 schematically illustrates fluid transfer probe vacuum drying station according to one embodiment of the invention.

FIG. 19A schematically shows a fluid transfer probe washing station from a perspective view according to one embodiment of the invention.

FIG. 19B schematically depicts another fluid transfer probe washing station from a perspective view according to one embodiment of the invention.

FIG. 20A schematically illustrates a wash reservoir that includes a transparent perspective view of a non-pressure-based fluid transfer probe mount according to one embodiment of the invention.

FIG. 20B schematically shows a non-pressure-based fluid transfer probe mounted on a non-pressure-based fluid transfer probe mount from a perspective view according to one embodiment of the invention.

FIG. 21 is a block diagram showing a representative fluid transfer probe washing station according to one embodiment of the invention.

### DETAILED DISCUSSION OF THE INVENTION

I. Definitions

Before describing the present invention in detail, it is to be understood that this invention is not limited to particular devices, systems, or methods, which can, of course, vary. It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which the invention pertains. In describing and claiming the present invention, the following terminology and grammatical variants will be used in accordance with the definitions set out below.

An “array” refers to an ordered, regular, or spatially defined pattern, grouping, or arrangement of components. For example, an array of pins disposed in a pin tool head of a pin tool of the invention typically includes a spatially defined pattern of pins of essentially any number (e.g., 2, 4, 6, 12, 24, 48, 96, 192, 384, 1536, 3456, 9600, or more pins). For a given number of pins or wells disposed in a multi-well plate, alternative spatial patterns are typically possible. To illustrate, a 1536-well plate optionally includes an array of 32 rows by 48 columns of wells (i.e., a 32×48 array), a 24×64 array, or the like. In preferred embodiments, arrays of pins, wells, or the like have footprints that correspond to arrays of wells in commercially available or otherwise standard micro-well plates or other sample containers (e.g., 6 wells in a 3×2 array, 12 wells in a 3×4 array, 24 wells in a 6×4 array, 48 wells in a 6×8 array, 96 wells in a 8×12 array, 1536 wells is a 32×48 array, etc.).

A “footprint” refers to the area on a surface covered by or corresponding to a device component or portions thereof. For example, the pins of a pin tool head of the invention typically correspond to (e.g., fit into, match, align with, etc.) wells in a selected micro-well plate or other sample container. In addition, a footprint of a system component, such as a container station or nest, etc., also typically substantially corresponds to a footprint of such micro-well plates such that the micro-well plate fits into or aligns with, e.g., the container station.

The phrase “in sensory communication” with a particular region or component refers to the placement of, e.g., an analytic component in a position such that the analytic component is capable of detecting or analyzing a property of the region or component, a portion of the region or component, or the contents of the region or component or a portion of the region or component, for which the analytic component is intended. In certain embodiments, for example, fluid sensors are disposed in sensory communication with components of fluid transfer probe washing stations.

The term “non-pressure-based fluid transfer probe” refers to device that is capable of transferring aliquots of fluid from a fluid source to a fluid destination without drawing the aliquots from the fluid source under an applied pressure. For example, fluid aliquots typically adhere to a non-pressure-based fluid transfer probe such that the probe can transfer the aliquots to the destination, e.g., the wells of a multi-well plate. To further illustrate, a non-pressure-based fluid transfer probe typically includes at least one pin. In preferred embodiments, a non-pressure-based fluid transfer probe includes a pin tool having multiple pins. Typically, the pins of a pin tool have a footprint that corresponds to the wells of a multi-well container, such as a standard microtiter plate, so that the pins can access the wells to deposit fluid volumes into or withdraw fluid volumes from the wells substantially simultaneously. More specifically, pin tools typically have, e.g., 6, 12, 24, 48, 96, 192, 384, 768, 1536, 3456, 9600, or more pins. An example of a pressure-based fluid transfer device is a pipetting device that draws fluid into pipette tips under pressure.

“Electromagnetic radiation” refers to a form of transmitted energy that exhibits both wave and particulate properties. “Source electromagnetic radiation” refers to electromagnetic radiation that is transmitted from an electromagnetic radiation source, such as a laser, a laser diode, an electroluminescence device, a light-emitting diode, an incandescent lamp, an arc lamp, a flash lamp, a fluorescent lamp, or the like. “Sample electromagnetic radiation” refers to electromagnetic radiation that is transmitted from one or more samples, such samples disposed in the wells of a multi-well assay plate, samples disposed on the surface of a support, or the like. Exemplary types of sample electromagnetic radiation include fluorescence and luminescence.

II. Description of Exemplary Embodiments

A. Systems Overview

While the present invention will be described with reference to a few specific embodiments, the description is illustrative of the invention and is not to be construed as limiting the invention. Various modifications to the present invention can be made to the preferred embodiments by those skilled in the art without departing from the true scope of the invention as defined by the appended claims. It is noted here that for a better understanding, like components are designated by like reference letters and/or numerals throughout the various figures.

The present invention provides sample assaying systems that can support a broad range of assay formats, including screens for compounds with desired properties. The systems of the invention are typically highly automated with minimal user intervention for repeated usage at high throughput in, e.g., laboratory and industrial settings. The systems described herein are also highly adaptable such that a variety of samples and sample assays can be accommodated by the systems to acquire information about the samples.

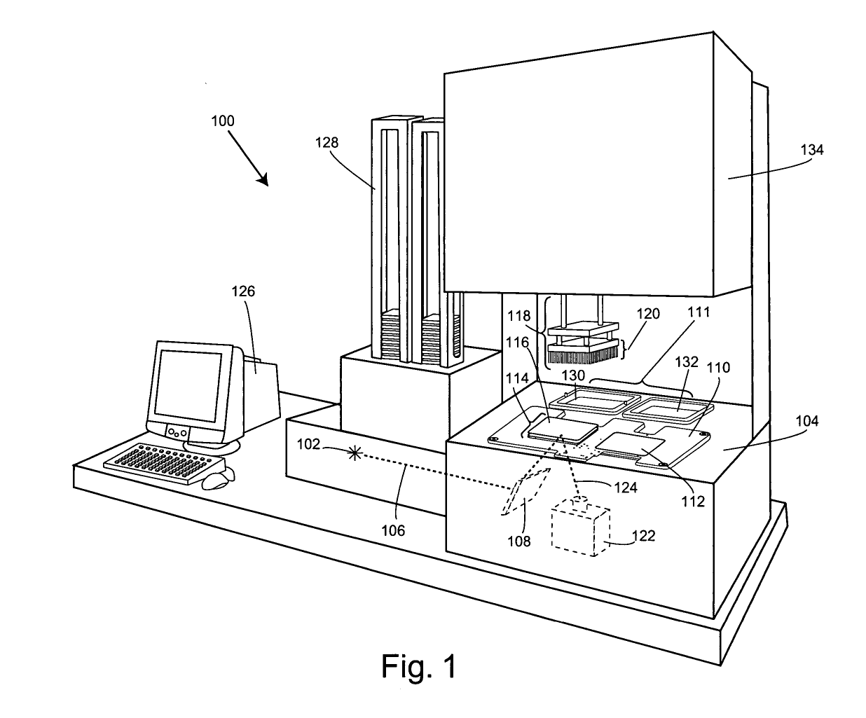

FIG. 1 schematically illustrates a sample assaying system from a perspective view according to one embodiment of the invention. As shown, system 100 includes electromagnetic radiation source 102, which is schematically depicted as a laser. Other electromagnetic radiation sources are also optionally adapted for use in the systems of the invention, including electroluminescence devices, laser diodes, light-emitting diodes (LEDs), incandescent lamps, arc lamps, flash lamps, fluorescent lamps, and the like. System 100 also includes sample assaying region 104, which is configured to receive source electromagnetic radiation 106 from electromagnetic radiation source 102 via mirror 108. Various optical systems are optionally utilized or adapted for use in the systems of the invention. Exemplary optical systems are described or referred to herein. Other suitable optical systems are known in the art and will be apparent to those of skill.

In preferred embodiments, sample assaying region 104 includes container positioning device 110, which includes container stations 112 and 114 that are each structured to position container 116 (shown as a multi-well container) relative to fluid transfer device 118. Fluid transfer device 118 includes non-pressure-based fluid transfer probe 120 (shown as a pin tool). Sample assaying region 104 also includes transfer probe washing station 111, which includes wash reservoirs 130 and 132 for washing non-pressure-based fluid transfer probe 120. Fluid transfer device 118 is configured to transfer fluid in at least one selected region (e.g., sample assaying region 104, as shown) of system 100. In preferred embodiments, non-pressure-based fluid transfer probe 120 is removably attached to a chassis of fluid transfer device 118. As also shown, system 100 also includes detector 122 configured to detect sample electromagnetic radiation 124 received from sample assaying region 104. Various detectors are optionally adapted for use in the systems of the invention including, e.g., charge-coupled devices (CCDs), intensified CCDs, photomultiplier tubes (PMTs), photodiodes, avalanche photodiodes, etc. Hood 134 of system 100 moves to enclose sample assaying region 104 to exclude, e.g., electromagnetic radiation other than source and sample electromagnetic radiation 106 and 124, respectively, or other contaminates that may bias assay results from sample assaying region 104.

System 100 also includes controller 126 (shown as computer) that is typically operably connected to, e.g., electromagnetic radiation source 102, fluid transfer device 118, and detector 122. Optionally, controller 126 is also operably connected to other system components. The controllers of the invention typically include at least one logic device (e.g., a computer such as the one illustrated in FIG. 1) having one or more logic instructions that direct operation of one or more components of the system. Also shown is container storage component 128, which stores containers before and/or after being assayed. Other components such as container incubation components and robotic devices among others are also optionally included in the systems of the invention. All of these system components are described in greater detail below.

B. Non-Pressure-Based Fluid Transfer Probes and Fluid Transfer Devices

One of the significant advantages of the present invention is the reproducible transfer of fluids at higher levels of throughput than can be achieved with more conventional systems such as those which rely solely upon pressure-based methods of fluid transfer. For example, pipette tips commonly used in various pipetting devices often become completely or partially obstructed which can yield inaccurate delivery of selected fluid volumes, if at all, which ultimately may bias assay results. In addition, assays or screens performed utilizing these types of pressure-based devices often necessitate replacing pipette tips at various steps in the particular protocol, which further limits the throughput of the assay being performed. Furthermore, the cost of disposable pipette tips can significantly add to the overall cost of running a large number of assays. Although pressure-based fluid transfer devices are also optionally used in the systems described herein, the present invention can avoid the shortcomings of these devices by utilizing non-pressure-based fluid transfer probes to effect reliable fluid transfer.

In preferred embodiments, the non-pressure-based fluid transfer probes used in the systems of the invention are pin tools. The pins tools utilized in these systems generally include a support structure having at least one attachment feature that can removably attach the pin tool to a chassis or other structural component of the fluid transfer device of the system. Attachment features can be in the form of hooks or hook mounts that hook unto corresponding components of the chassis. Any other functionally equivalent attachment feature can also optionally be utilized or adapted for use in the systems described herein. In addition, the pin tools of the invention also include pin tool heads that have at least one pin attached to the head. Pins are typically free floating in pin tool heads or resiliently coupled to pin tool heads by a resilient coupling, such as a spring, an elastomer, or other such coupling device or material known in the art, to minimize the risk of damaging a component of the system and/or a sample container or support if a pin contacts the container or support. Pin tool heads are typically removably attached to the support structures of pin tools. This facilitates exchanging, e.g., pin tool heads having different pin densities and/or configurations, etc. Pin tool heads are generally removably attached to the support structure by one or more attachment components, such as set screws, spring ball sockets, and/or the like. In preferred embodiments, pin tool heads further include a rotational adjustment feature (e.g., a screw or the like) such that pin tool heads are capable of rotating relative to corresponding support structures, e.g., to align the pin tool heads with various containers or supports and/or various system components. Rotational adjustment features or mounts are described in greater detail below.

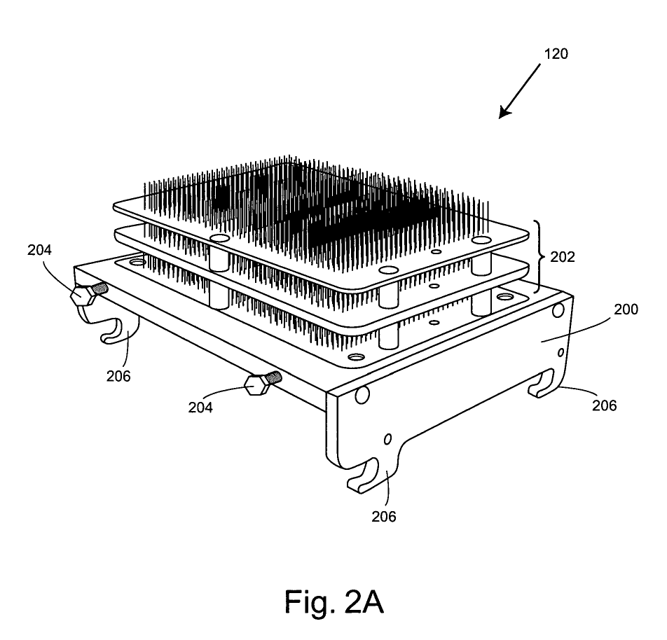

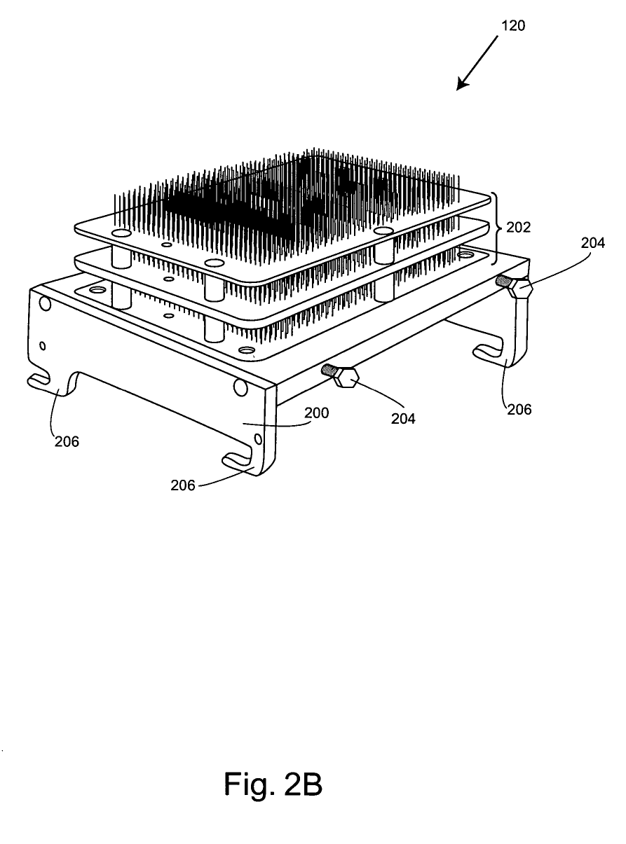

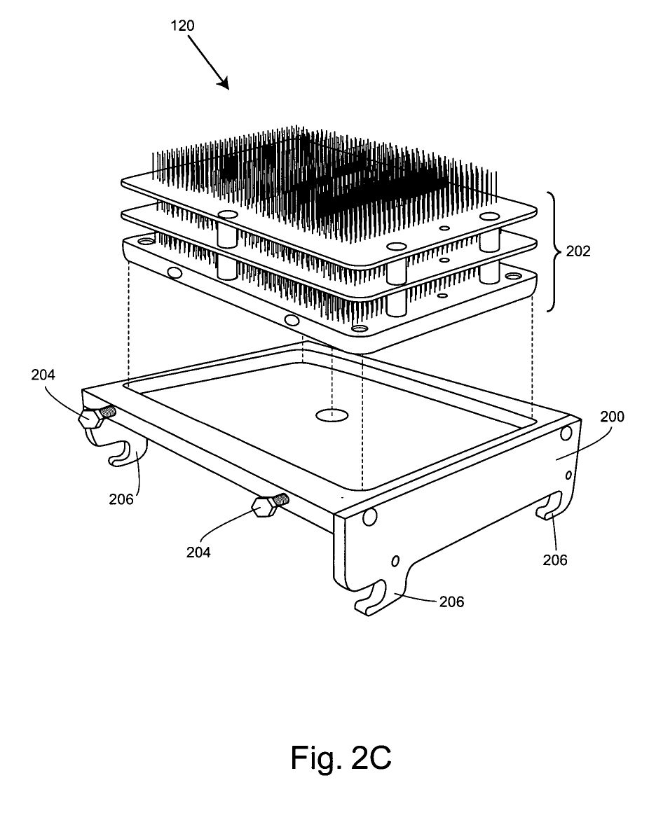

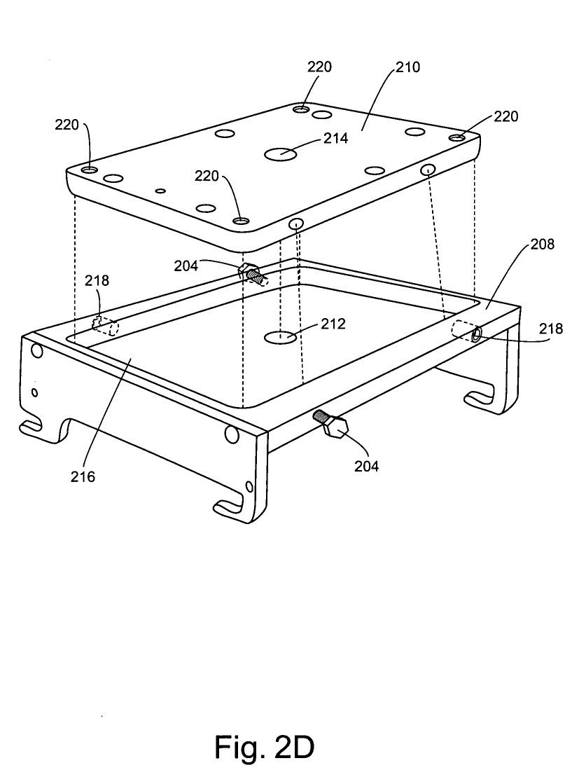

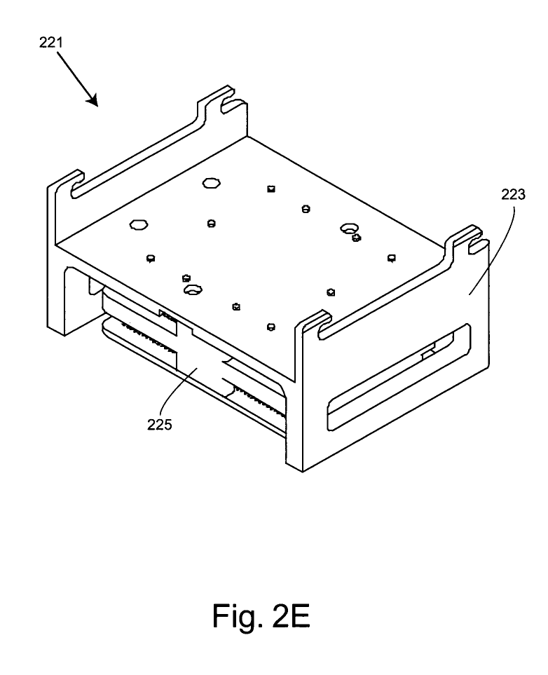

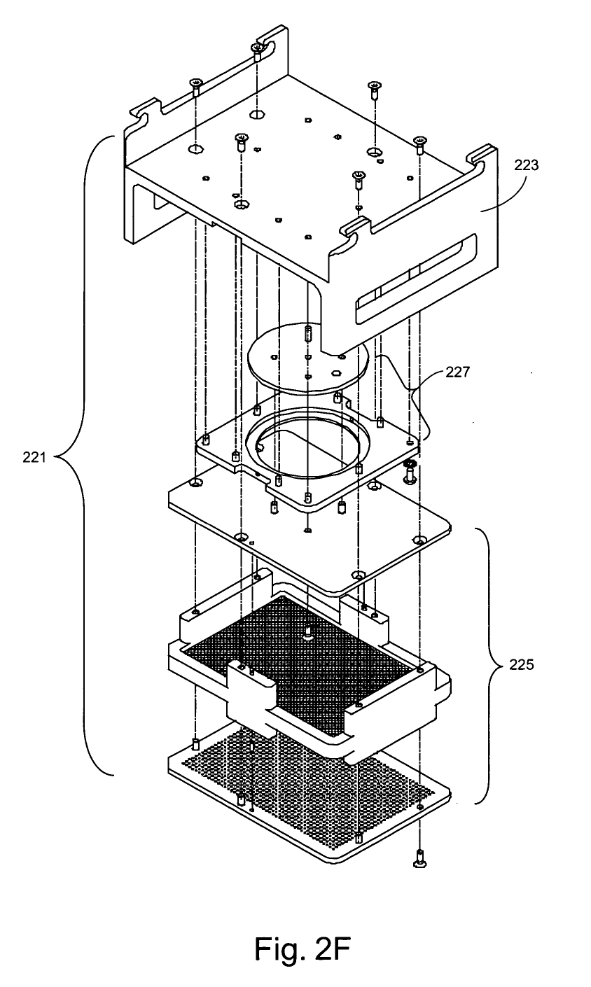

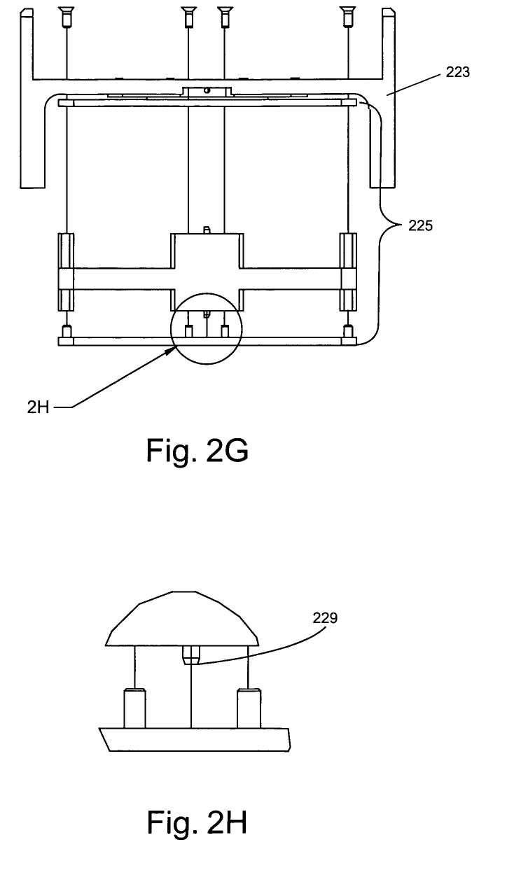

FIGS. 2A-C schematically show pin tool 120 from various perspective views according to one embodiment of the invention. As shown, pin tool 120 includes support structure 200 and pin tool head 202. Pin tool head 202 is removably attached to support structure 200 by set screws 204. Pin tool heads typically include a mounting plate and one or more floating fixtures or plates. As also shown, support structure 200 also includes hooks 206, which removably attach support structure 200 to another component of the fluid transfer device, such as the chassis of the fluid transfer device, which is described further below. Pin tool head 202 includes 1536 pins in a 32×48 array that has a footprint corresponding to 1536-well micro-well plate. The pin tool heads of the invention optionally include other array configurations and/or numbers of pins to transfer fluid samples to and/or remove such fluid samples from selected multi-well containers or support surfaces. In preferred embodiments, pin tool heads of the systems described herein include numbers of pins that correspond to the number of wells in various standard multi-well plates, such as those having, e.g., 6, 12, 24, 48, 96, 192, 384, 768, 1536, 3456, 9600, or more wells. A wide variety of pin tools and pins are optionally used in the systems of the invention and some are commercially available from sources, such as V&P Scientific, Inc. (San Diego, Calif.), Beckman Coulter, Inc. (Fullerton, Calif.), Perkin Elmer Life Sciences (Boston, Mass.), and the like. Pins, for example, can be of varied lengths selected, e.g., according to the depth of the containers to be accessed. Pins can also have various cross-sectional dimensions (e.g., diameters, etc.) and be slotted, solid, etc. or otherwise varied according to the fluid volumes to be transferred. Pins can also be uncoated, or coated with, e.g., hydrophobic or lipophobic coating to provide additional control over the transfer of various types of solutions (e.g., organic or aqueous solutions).

In preferred embodiments, the pin tools of the invention include low profile rotational adjustment features or mounts. Conventional pin tools lack an intrinsic mechanism to adjust for the rotational axis of the pin tool. Instead, conventional devices are typically coupled to a separate rotational mount. A significant advantage of the pin tools of the invention is that a low profile rotational adjustment is generally built into the pin tools themselves, thereby eliminating the need for separate rotational mounts. This is schematically illustrated in FIG. 2D, which shows pin tool support structure 208 and top plate 210 of a pin tool head (floating plates and pins are not shown) from an exploded perspective view according to one embodiment of the invention. Pin tool support structure 208 and top plate 210 each include center holes 212 and 214, respectively, which align with one another when top plate 210 is positioned in top plate inset region 216 of pin tool support structure 208. Center holes 212 and 214 are each typically threaded to receive a center screw (not shown), which can be used to adjust the rotational axis of an attached pin tool head. Other functionally equivalent components aside from center screws (e.g., posts, ball and socket joints, etc.) can also be adapted for use as rotational adjustment features of the pin tools of the invention. In the embodiment shown in FIG. 2D, pin tool support structure 208 also includes spring tension devices 218 (e.g., spring ball sockets, etc.) apposed by set screws 204 to rotate the pin tool head about the center screw. In other embodiments, support structures include only set screws 204. This is shown, for example, in FIGS. 2A-C. Holes 220 are typically included to attach top plate stand-off components (e.g., flexed metal or polymeric strips, springs, elastomers, etc.) that resiliently couple a pin tool head to pin tool support structure 208. Optionally, top plate stand-off components are not included or are attached to pin tool support structure 208.

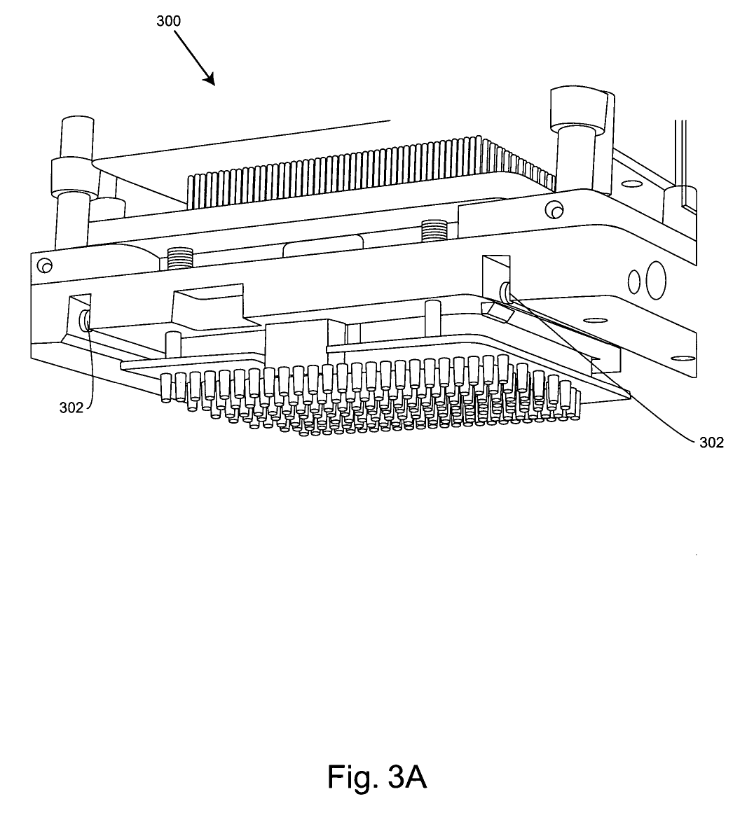

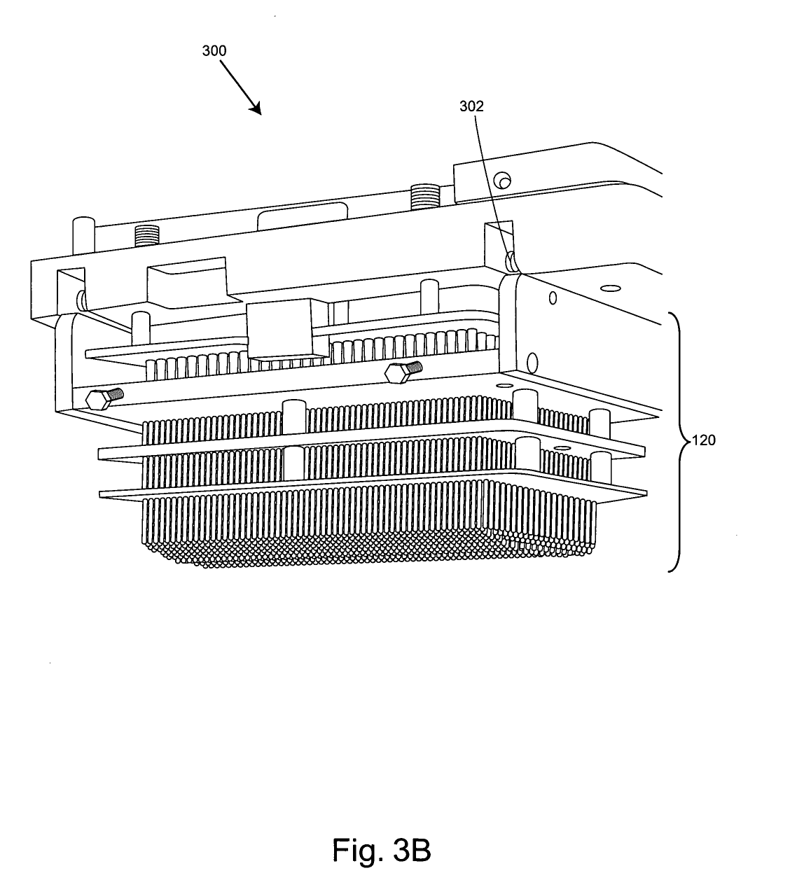

Pin tools typically removably attach to other components of the fluid transfer devices of the assaying systems by various attachment features, including the hook mounts described above. In certain embodiments, for example, pin tools removably attach a chassis of a pressure-based fluid transfer device (e.g., a pipetting system, etc.) to afford the user the option of using either a pin tool or pipettes to transfer fluids between various types of containers and/or supports. FIG. 3A schematically shows a chassis of a fluid transfer device that includes such a pipetting system. As shown, chassis 300 includes horizontal posts 302 (two are not within view) to which hooks 206 of pin tool 120 are capable of being attached. FIG. 3B schematically depicts pin tool 120 attached to chassis 300 via horizontal posts 302. When pin tools are not attached to fluid transfer device chassis, they are optionally disposed in a docking station. In certain embodiments, for example, wash stations of the invention can also function as docking stations for pin tools. Docking and wash stations are described in greater detail below. In some embodiments of the invention, fluid transfer devices do not include pipetting systems in addition to the capability of using pin tools to effect fluid transfer. In these embodiments, at least pin tool support structures are optionally manufactured as non-removable components of fluid transfer devices.

To further illustrate, FIGS. 2E and F schematically illustrate another exemplary pin tool according to one embodiment of the invention. More specifically, FIG. 2E schematically shows pin tool 221 from a perspective view, while FIGS. 2F and G schematically depict pin tool 221 from exploded perspective and exploded front views, respectively. As shown, pin tool 221 includes support structure 223 and pin tool head 225 (pins not shown). Pin tool 221 also includes rotational adjustment feature 227 (shown as a rotation stage and as a rotation stage capture block). FIG. 2H schematically shows an interface between components of pin tool head 225 from pin tool 221 from a detailed front view. The interface includes dowel pin 229, which is received by an opposing hole (not within view) when pin tool head 225 is assembled.

The fluid transfer devices of the invention typically include robotic translation systems (e.g., X-Y-Z translations systems, etc.) that move pin tools relative other components of the system. In certain embodiments, for example, a fluid transfer device lowers a pin tool such that the pins contact fluidic samples in a multi-well sample compound plate. The fluid transfer device then typically withdraws from the compound plate such that fluid adheres to the pins of the pin tool and translocates the pin tool such that the fluidic samples volumes adhered to the pins are dispensed into corresponding wells in a multi-well sample assay plate for analysis, e.g., excitation by the electromagnetic radiation from the electromagnetic radiation source and detection of sample electromagnetic radiation from the assay plate by the detector. Robotic translations systems are typically operably connected to controllers of the assay systems, which controller generally includes one or more computers or other logic devices having system software that directs the operation of the translation systems. Controllers are described in greater detail below.

C. Sample Assaying Regions, Container Positioning Devices, and Fluid Transfer Probe Washing and Drying Stations

The sample assaying regions of the systems of the invention are configured to receive source electromagnetic radiation from the electromagnetic radiation source. In preferred embodiments, sample assaying regions also include container positioning devices that position containers relative to the fluid transfer device and/or the detector. Sample assaying regions optionally further include fluid transfer probe washing stations to wash fluid transfer probes before and/or after selected fluid transfer processes, and fluid transfer probe drying stations (e.g., blotting stations, vacuum drying stations, etc.) to dry fluid transfer probes as desired.

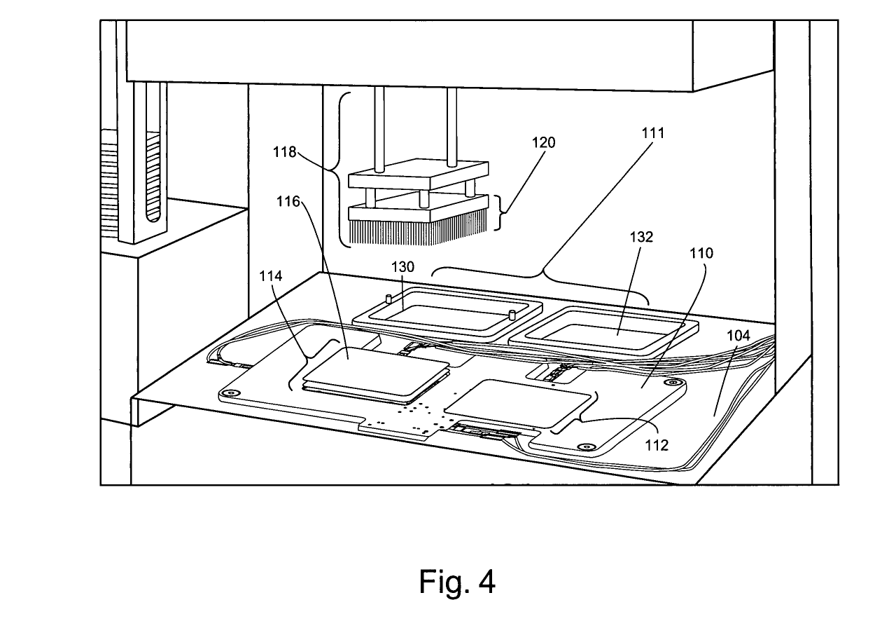

FIG. 4 schematically shows sample assaying region 104 from a perspective view according to one embodiment of the invention. As shown, sample assaying region 104 includes container positioning device 110, which includes container stations 112 and 114 that are each structured to position containers relative to fluid transfer device 118. In preferred embodiments, container stations 112 and 114 that are structured to position multi-well plates. For example, container station 112 is optionally utilized to position a multi-well plate containing sample compounds and container station 114 is optionally utilized to position an assay multi-well plate into which compounds are transferred from the sample compound multi-well plate positioned in container station 112 using fluid transfer device 118. As also shown in this embodiment, sample assaying region 104 additionally includes fluid transfer probe washing station 111. Certain assay protocols include washing pin tool 120 in one or both wash reservoirs 130 and 132 before and/or after performing a particular transfer step. Optionally, wash reservoir 130 is also used as a docking station to position pin tool 120 when it is detached from the chassis of fluid transfer device 118. In certain embodiments, fluid transfer probe washing stations are not included in the assaying systems of the invention or are located in a region other than sample assaying region 104. In some embodiments, for example, one or both of reservoirs 130 and 132 are replaced by fluid transfer probe blotting stations or vacuum drying stations, which effect the removal of fluids that adhere to the pins of pin tool 120. Each of these system components is described in greater detail below.

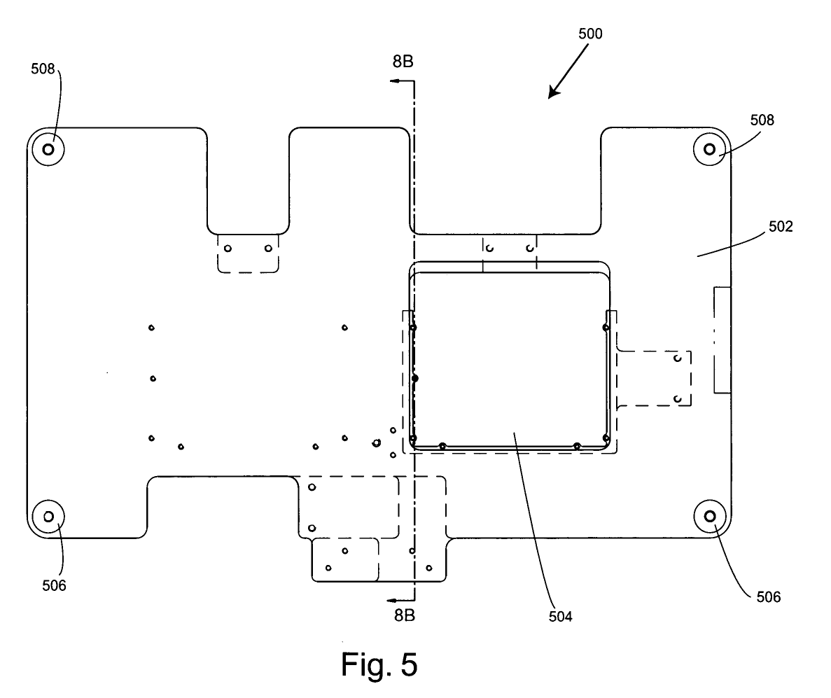

In preferred embodiments, the sample assaying regions of the systems described herein include container positioning devices, e.g., to position sample containers relative to fluid transfer devices. FIG. 5 schematically depicts support structure 502 of container positioning device 500 from a bottom view. As shown, support structure 502 includes cutout or orifice 504 disposed through container positioning device 500 such that when an assay container is positioned over orifice 504, the container can receive electromagnetic radiation from an electromagnetic source (e.g., via an optical system, etc.) and/or the detector can receive electromagnetic radiation from the container through orifice 504. Although other materials such as structural polymers, steel and other metals are optionally utilized, support structure 502 is typically fabricated from aluminum and finished with a black anodization. Component fabrication is described further below.

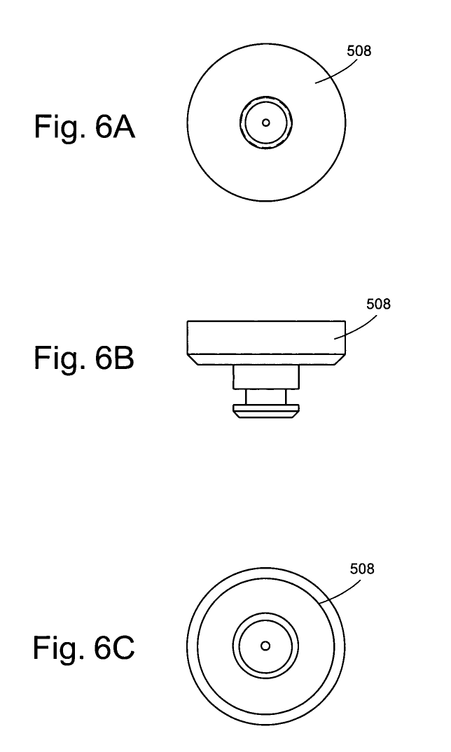

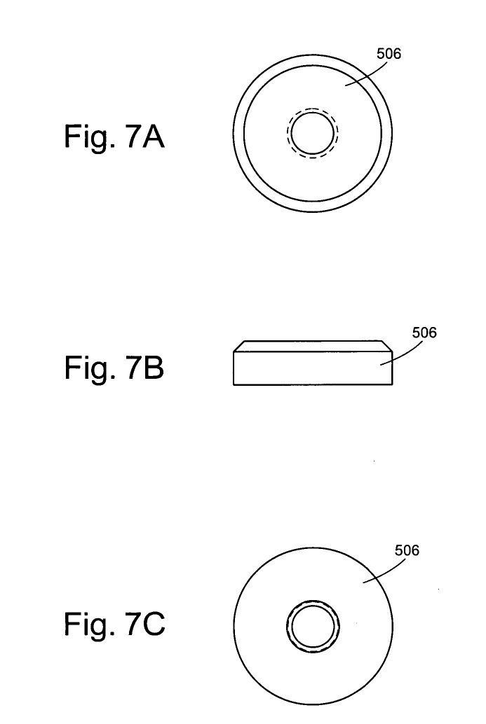

As also shown in FIG. 5, front feet 508 and rear feet 506 are typically attached to support structure 502 to position container positioning device 500 in the sample assaying region of a system of the invention. In certain embodiments, for example, a sample assaying region will include corresponding indentations that are configured to receive front feet 508 and rear feet 506 when container positioning device 500 is positioned in the system. FIGS. 6 and 7 schematically depict front feet 508 and rear feet 506, respectively, from various detailed views. In particular, FIGS. 6A-C schematically show front foot 508 from detailed bottom, side, and top views, respectively. FIGS. 7A-C schematically depict rear foot 506 from detailed top, side, and bottom views, respectively. While other materials are optionally utilized, front feet 508 and rear feet 506 are typically fabricated from aluminum and optionally finished with a black anodization.

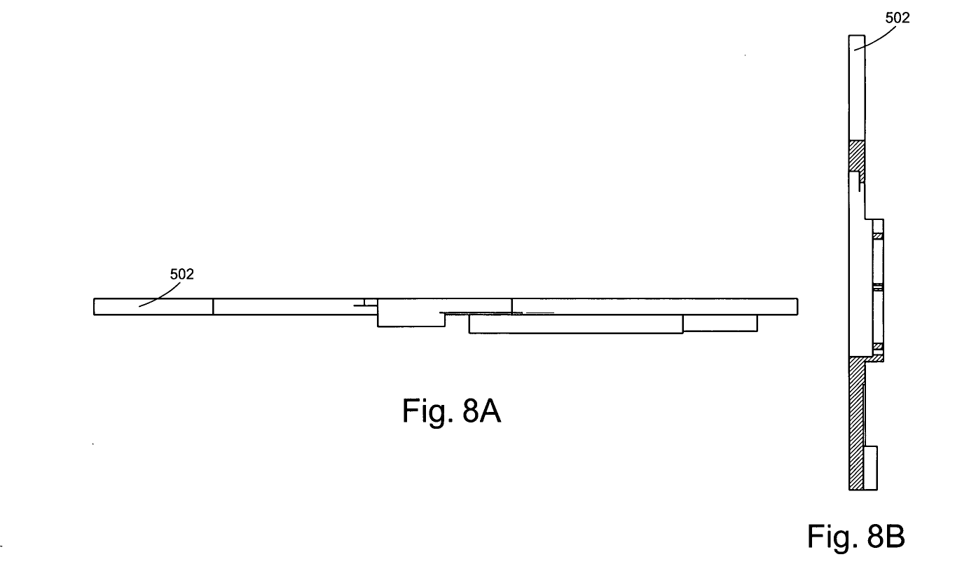

FIG. 8A schematically shows a side view of support structure 502 shown in FIG. 5. To further illustrate the container positioning devices of the invention, FIG. 8B schematically illustrates a cross-sectional side view along section 8B of support structure 502 depicted in FIG. 5.

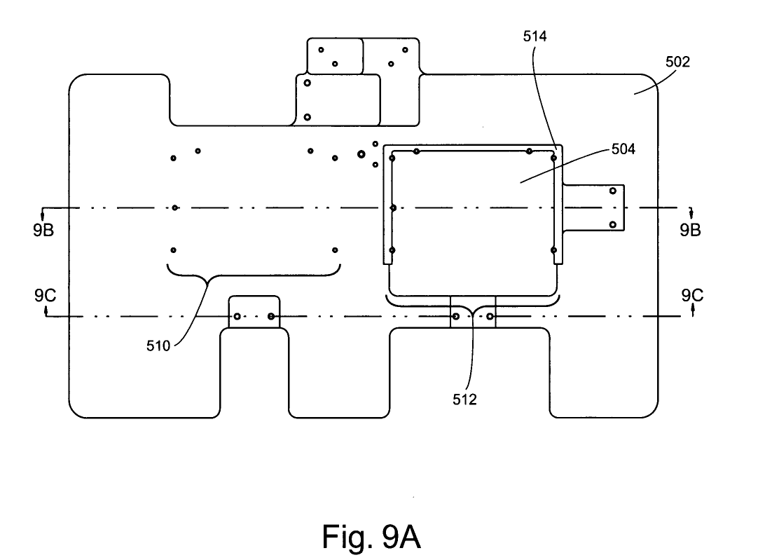

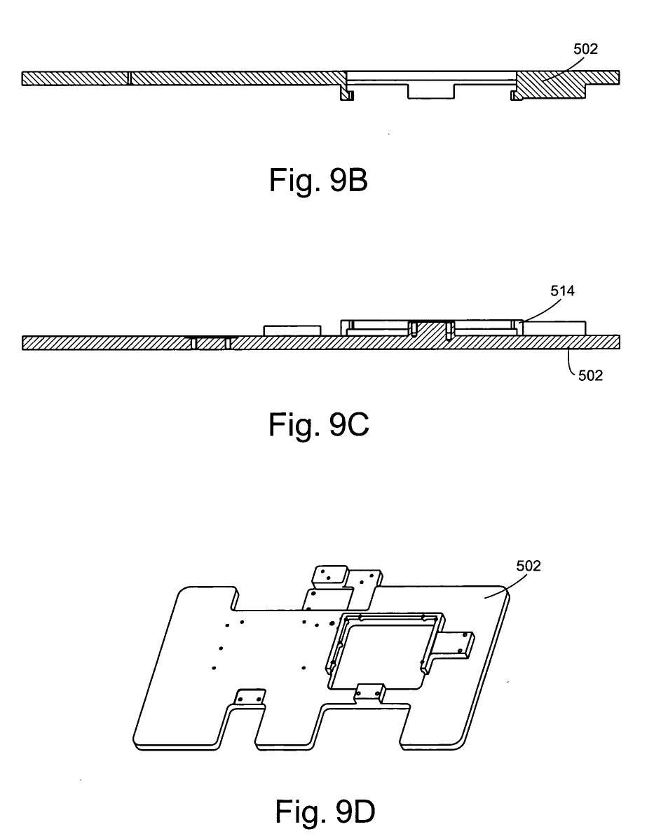

The container positioning devices of the invention generally include multiple container stations, e.g., to position multiple containers for fluid transfer when performing a given assay. In preferred embodiments, at least two of the container stations are tiered, that is, disposed at different levels. In systems that include robotic handlers, tiered container stations have the advantage of allowing a robotic handler to access and handle (e.g., grasp and re-locate) a first container positioned at one tiered container station without contacting a second container positioned at another tiered container station. This is further illustrated in, e.g., FIGS. 9A-D. In particular, FIG. 9A schematically shows support structure 502 shown in FIG. 5 from a top view. As shown, support structure 502 includes container station 510 and container station 512. Container station 512 includes orifice 504 disposed through support structure 502, as described above. In addition, container station 512 further includes tier structure 514 disposed around a portion of orifice 504. Tier structure 514 positions containers at a different level in container station 512 than those positioned in container station 510. FIGS. 9B and C schematically depict cross-sectional side views of support structure 502 shown in FIG. 9A along sections 9B and 9C, respectively. To further illustrate, FIG. 9D schematically illustrates support structure 502 from a top perspective view.

The container stations of the container positioning devices of the invention also optionally include heating elements (e.g., external to or integral with the container stations) to regulate temperature in the container or on the other support, e.g., when an assay is performed in the system. Suitable heating elements that can be adapted for use in the systems of the invention are generally known in the art and are readily available from various commercial sources. Heating elements are typically operably connected to system controllers, which control operation of the elements.

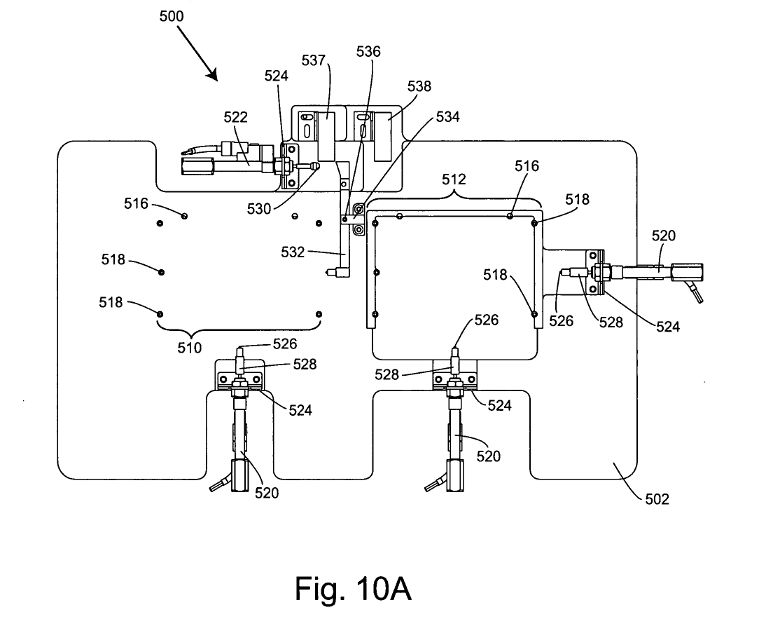

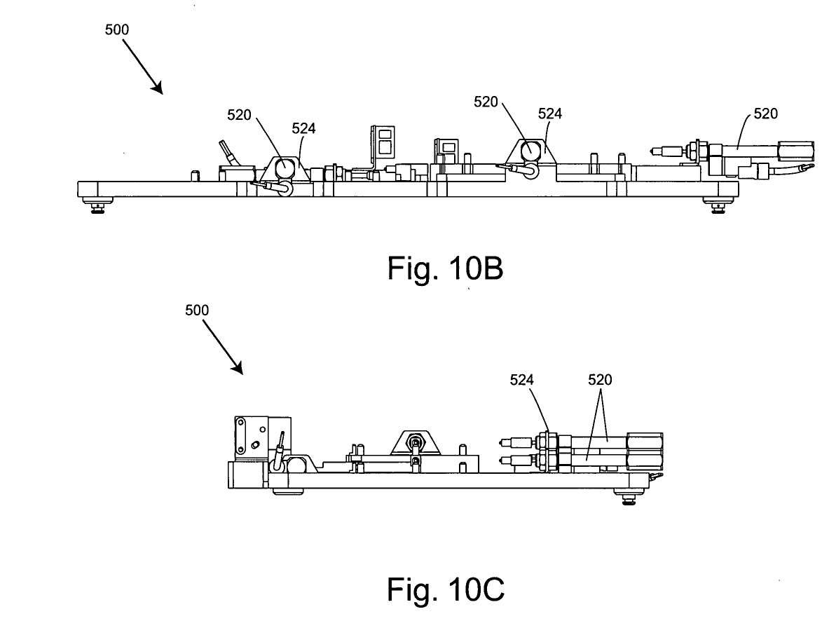

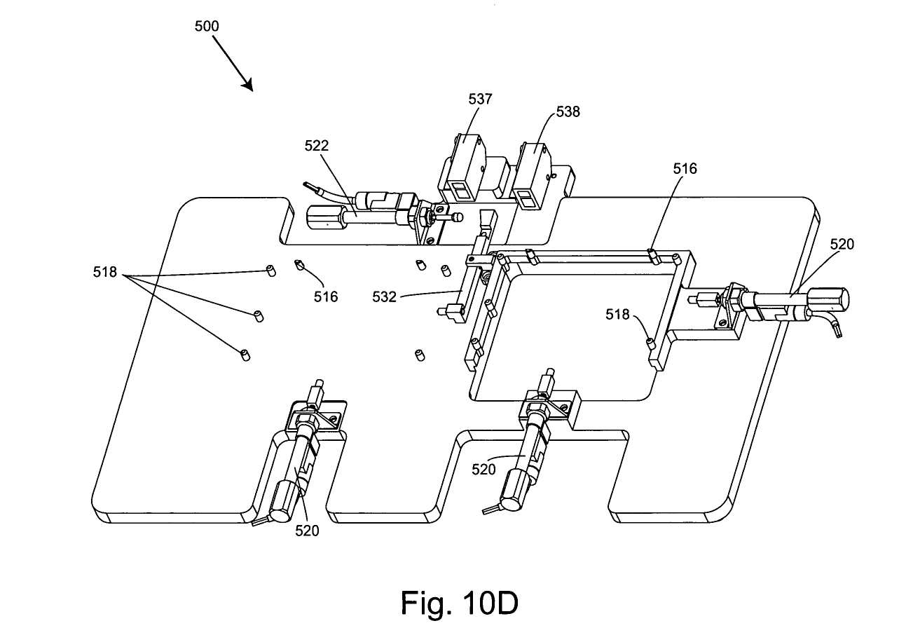

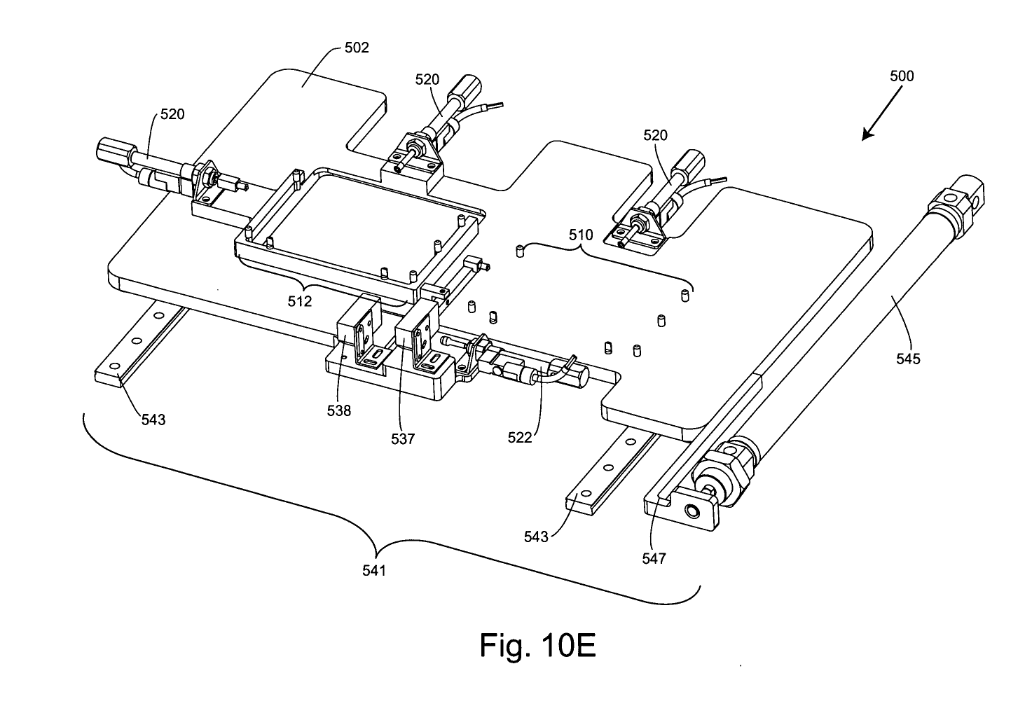

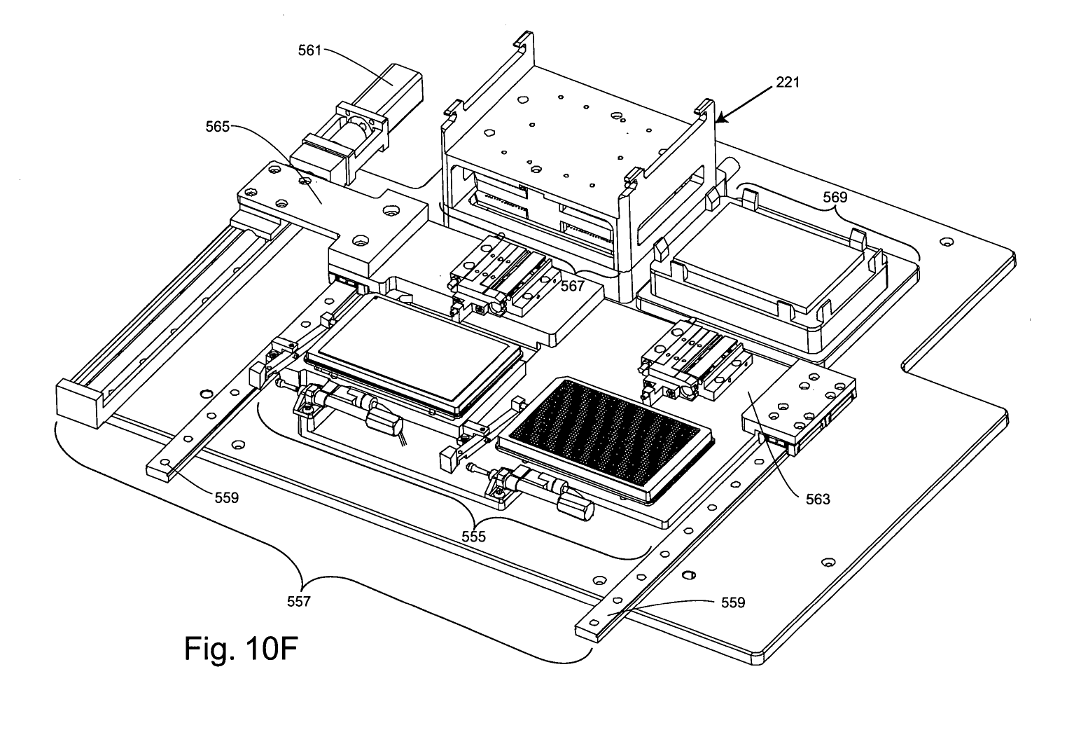

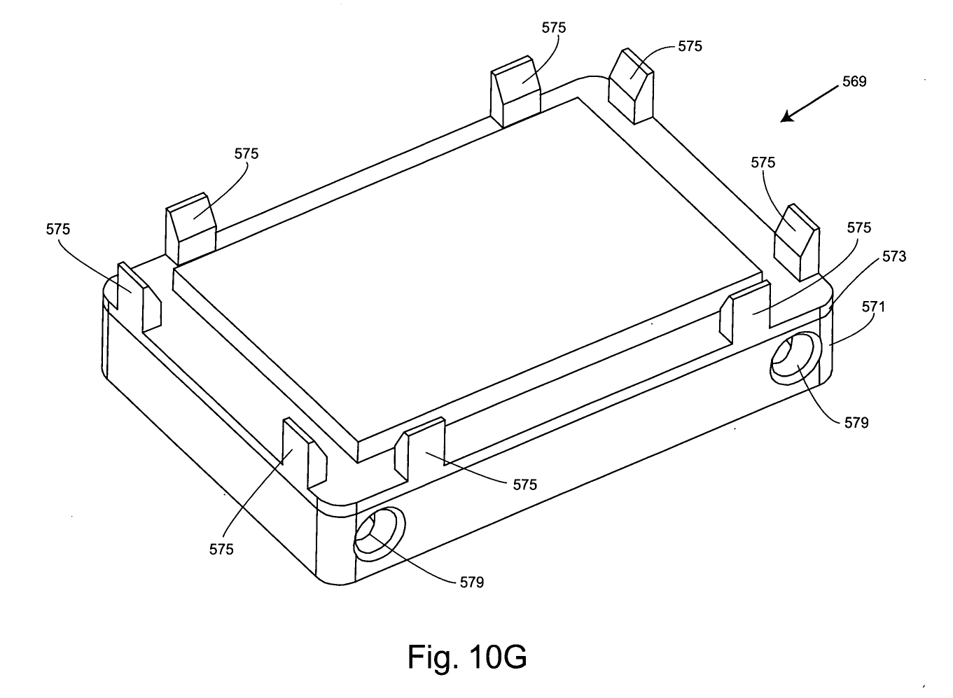

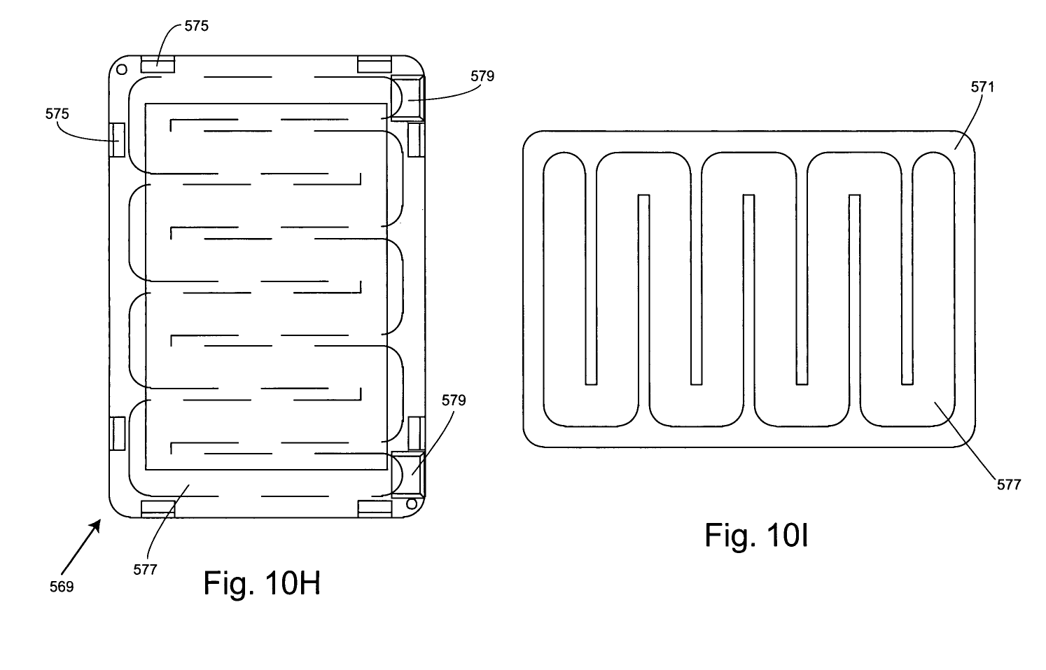

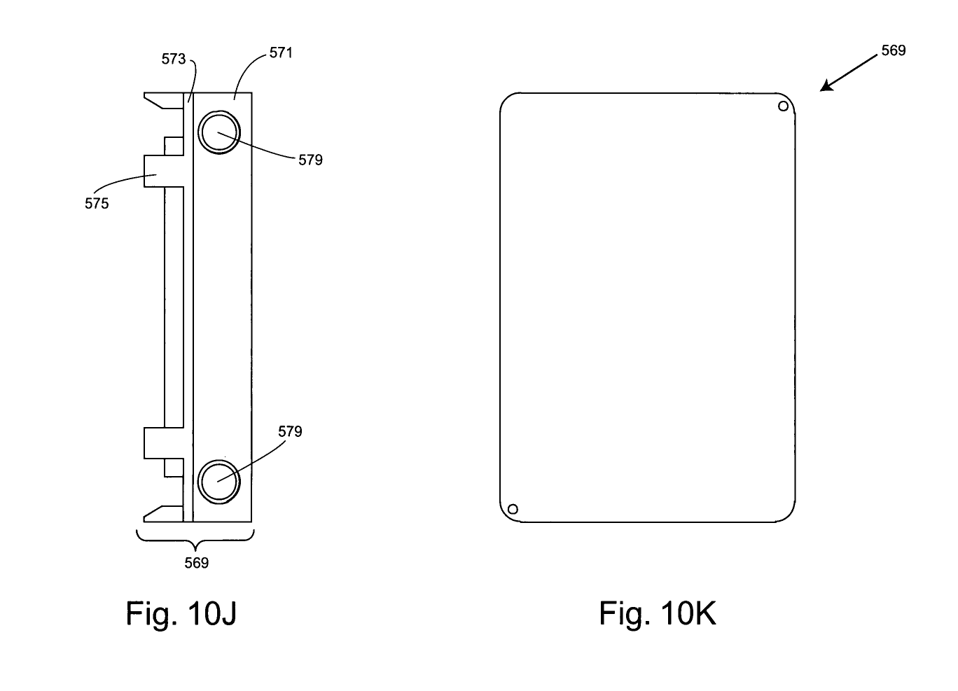

The container positioning devices of the invention generally include alignment members that are positioned to contact surfaces of containers when the containers are positioned in the container stations such that the containers align with the fluid transfer device. In addition, these container positioning devices also typically include pushers that push the containers into contact with the alignment members when the containers are positioned in the container stations. Embodiments of these aspects of the container positioning devices of the invention are illustrated in FIGS. 10A-D. More specifically, FIG. 10A schematically shows container positioning device 500 from a top view. As shown, container positioning device 500 includes alignment members 516 (shown as trimmed face pins) and alignment members 518 (shown as pins), which align with inner surfaces of standard multi-well plates positioned in container stations 510 and 512. As also shown, container positioning device 500 further includes pneumatically-driven pushers 520 and 522 (e.g., air cylinders or the like), which effect container positioning relative to alignment members 516 and 518. Pushers 520 and 522 are mounted to support structure 502 via pusher mounts 524 and are operably connected to pressure sources (not shown). Pushers 520 include spring plungers 526 and plunger posts 528. Pusher 522 includes knob 530 that contacts lever arm 532 to push lever arm 532 into contact with a container. Lever arm 532 is mounted to support structure 502 via pin capture block 534 and lever shaft 536. As also shown in FIG. 10A, container positioning device 500 also includes laser assemblies 537 and 538 for detecting the presence of containers in container stations 510 and 512, respectively. FIGS. 10B and C schematically show container positioning device 500 from side elevational views. In addition, FIG. 10D schematically illustrates container positioning device 500 from a perspective view.

To further illustrate aspects of the invention, FIG. 10E schematically shows a perspective view of container positioning device 500 of FIG. 10A mounted on translational mechanism 541. When container positioning devices are included in systems such as system 100 schematically shown in FIG. 1, translational mechanisms are optionally included such that container positioning devices can be translocated along at least one translational axis, e.g., to facilitate access to multi-well containers positioned in the container positioning devices by a user, a robotic translocation device, and/or the like. In the embodiment shown, translational mechanism 541 includes rails or tracks 543 on which container positioning device 500 is mounted and along which container positioning device 500 slides. In addition, actuator 545 (e.g., an air cylinder, motor, etc.) is operably connected to support structure 502 of container positioning device 500 via bracket 547. Actuator 545, which is generally operably connected to a controller, effects translocation of container positioning device 500 along tracks 543.

To further illustrate additional aspects of the invention, FIG. 10F schematically shows a perspective view of sample assaying region 553, which includes container positioning device 555 mounted on translational mechanism 557. As referred to above, translational mechanisms are optionally included so that container positioning devices can be translocated along at least one translational axis. In the embodiment shown, translational mechanism 557 includes rails or tracks 559 on which container positioning device 555 is mounted and along which container positioning device 555 slides. In addition, actuator 561 (e.g., an air cylinder, motor, etc.) is operably connected to support structure 563 of container positioning device 555 via bracket 565. Actuator 561, which is generally operably connected to a controller, effects translocation of container positioning device 555 along tracks 559.

As also shown in FIG. 10F, sample assaying region 553 also includes wash reservoir 567 and thermal modulation nest 569 according certain illustrative embodiments. Wash reservoirs and stations are also described further below. Thermal modulation nests are typically used to regulate temperatures in containers (e.g., compound plates, assay plates, etc.). To further illustrate, FIGS. 10G-K schematically depict various aspects of thermal modulation nest 569. More specifically, FIG. 10G schematically depicts thermal modulation nest 569 from a perspective view, FIG. 10H schematically shows thermal modulation nest 569 from a transparent top view, FIG. 101 schematically shows bottom plate 571 of thermal modulation nest 569 from a top view, FIG. 10J schematically illustrates thermal modulation nest 569 from a front view, and FIG. 10 schematically depicts thermal modulation nest 569 from a bottom view. As shown, thermal modulation nest 569 includes top plate 573 and bottom plate 571, which are generally attached (e.g., welded, bonded, adhered, etc.) to one another in an assembled device. Although other materials are optionally utilized, top plate 573 and bottom plate 571 are both fabricated from stainless steel in certain embodiments. Top plate 573 typically includes nest features 575 formed on a surface (e.g., via machining, molding, etc.), which are used to align containers on thermal modulation nest 569. Bottom plate 571 includes channel 577 (shown with a serpentine flow path), which communicates with orifices 579. Channels and orifices are typically formed by machining or other processes known to persons of skill in the art.

During operation, hoses are generally attached to orifices 579 and heated or cooled fluids are circulated through the hoses and channel 577 via orifices 579, e.g., to regulate temperatures in a container (e.g., a control plate or boat, etc.) disposed on thermal modulation nest 569. In certain embodiments, for example, the hoses are operably connected to a recirculated chiller unit (e.g., a NESLAB RTE-7 available from Thermo Electron Corporation (Newington, N.H.)). In these embodiments, the chiller unit typically cools a 50/50 ethylene-glycol and water mixture to 4° C. and circulates the fluid through thermal modulation nest 569. Typically, a drip tray or the like is positioned underneath thermal modulation nest 569 to catch condensate that forms on thermal modulation nest 569. Containers positioned on thermal modulation nest 569 are typically accessible by the pin tools described herein.

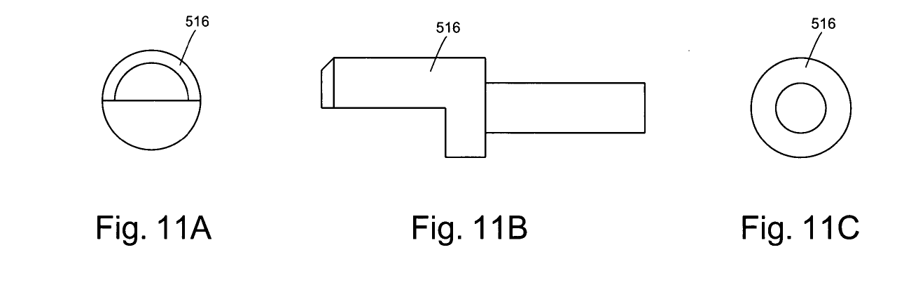

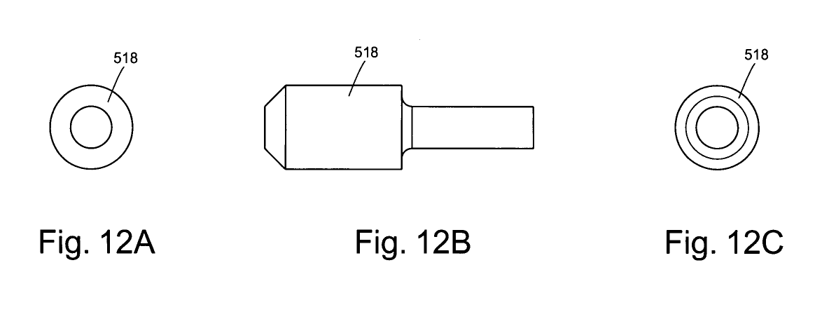

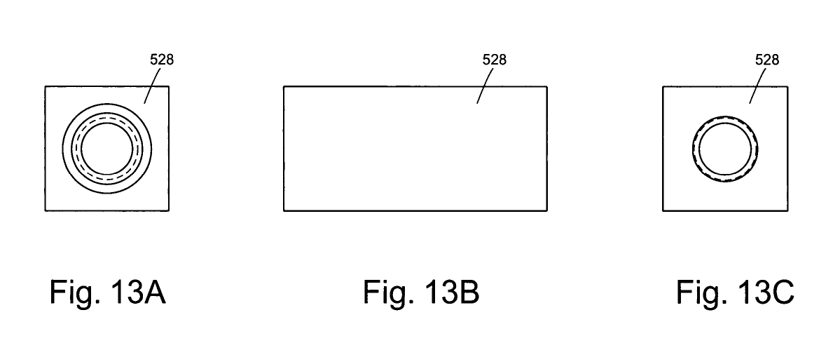

FIG. 11A schematically shows alignment member 516 of container positioning device 500 from a detailed top view, while FIGS. 11B and C schematically show alignment member 516 from detailed side and bottom views, respectively. Further, FIG. 12A schematically shows alignment member 518 of container positioning device 500 from a detailed top view, whereas FIGS. 12B and C schematically depict alignment member 518 from detailed side and bottom views, respectively. Additionally, FIGS. 13A-C schematically show plunger post 528 from detailed front, side, and rear views, respectively. Although other materials are optionally used, these components are typically fabricated from aluminum and optionally finished with a black anodization.

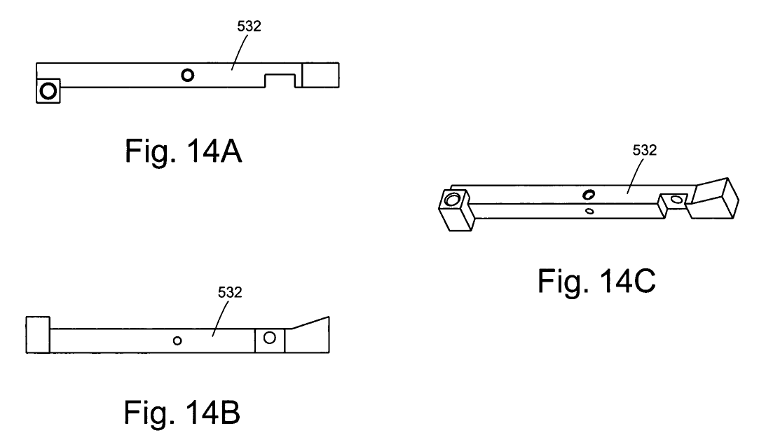

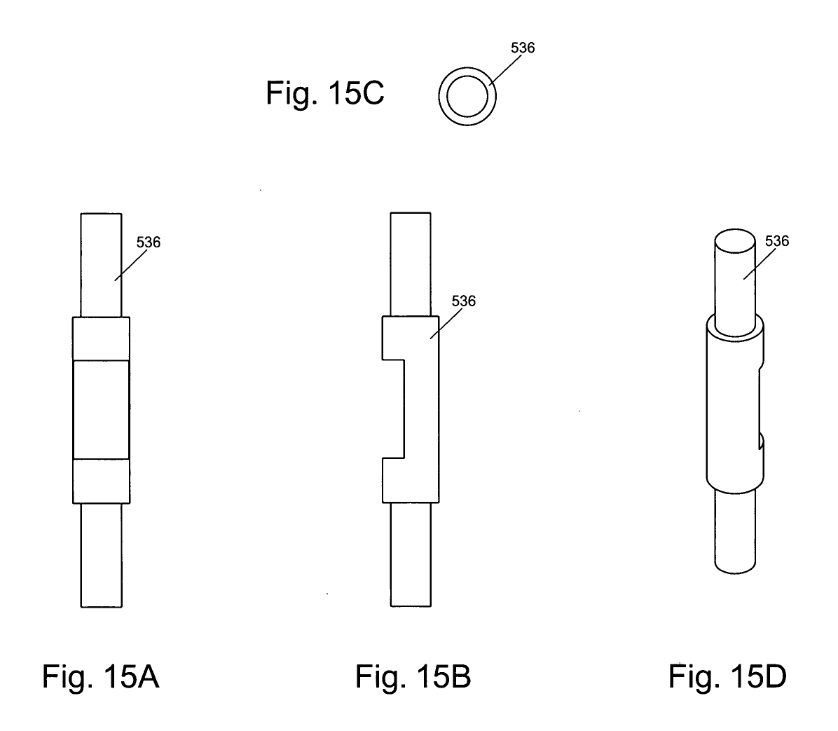

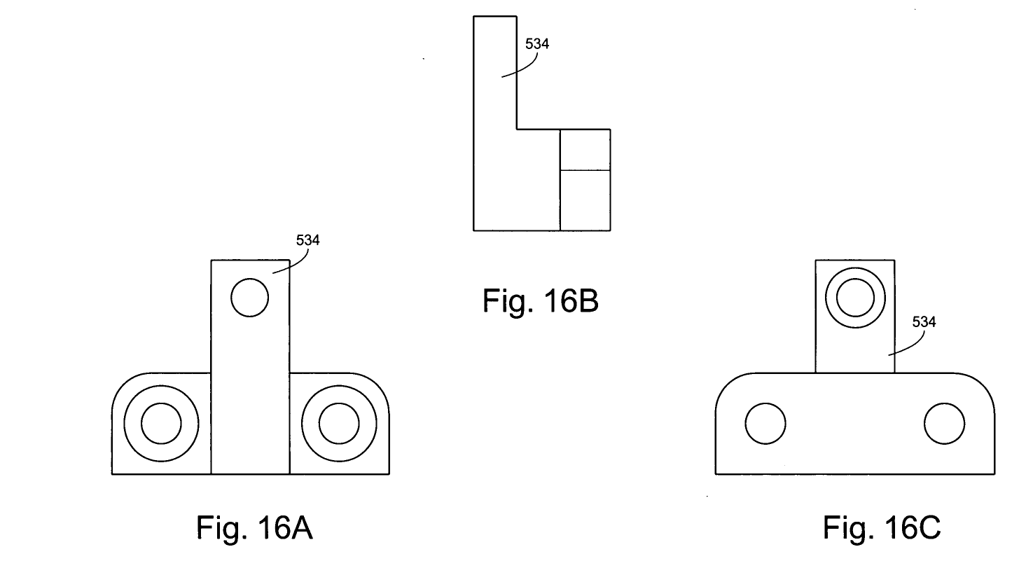

FIGS. 14-16 schematically show detailed views of various pusher components related to pusher 522. In particular, FIGS. 14A-C schematically show lever arm 532 from detailed front, rear, and perspective views, respectively. FIGS. 15A-D schematically depict lever shaft 536 from detailed front, side, top, and perspective views, respectively. In addition, FIGS. 16A-C schematically show pin capture block 534 from detailed top, side, and bottom views, respectively. As with other components of the container positioning devices of the invention, while other materials are optionally utilized, these components are also typically fabricated from aluminum and optionally finished with a black anodization.

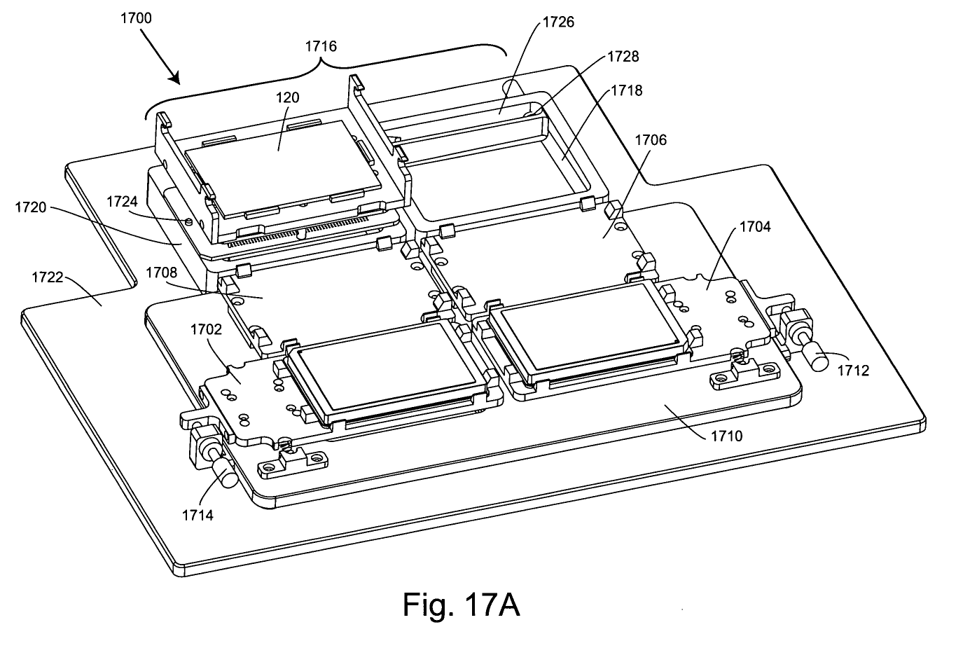

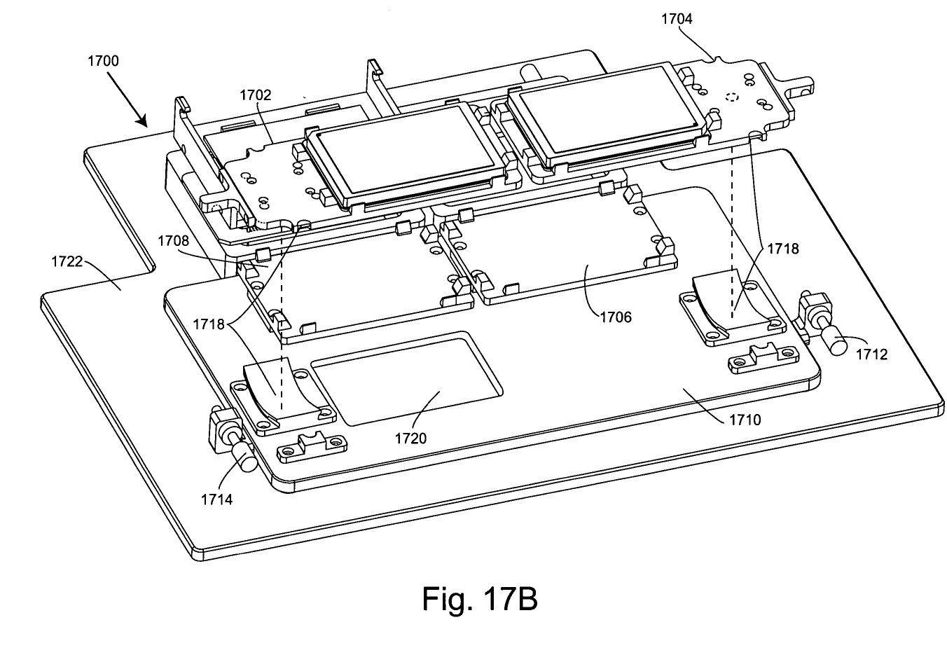

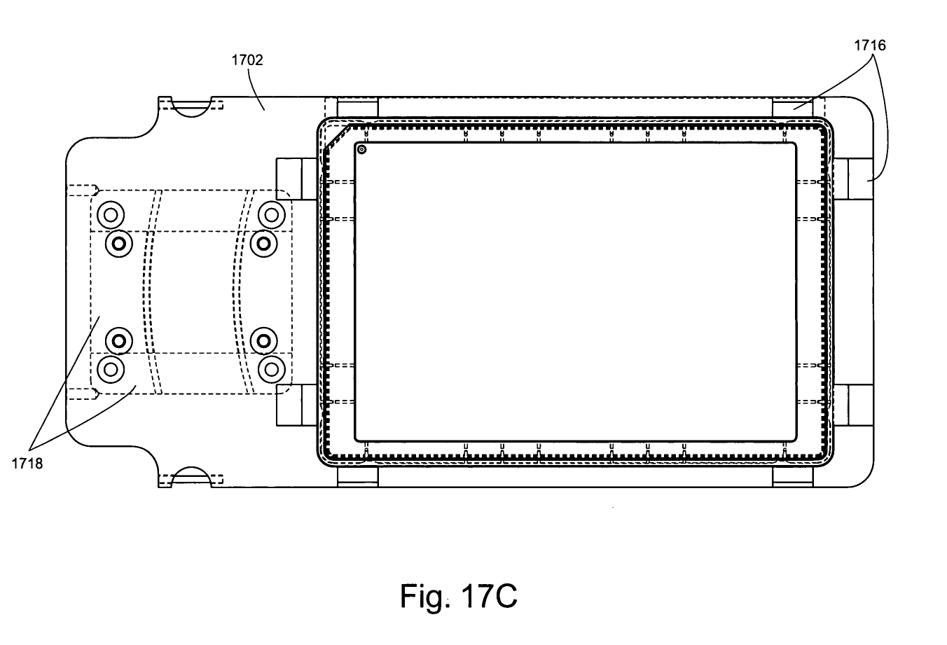

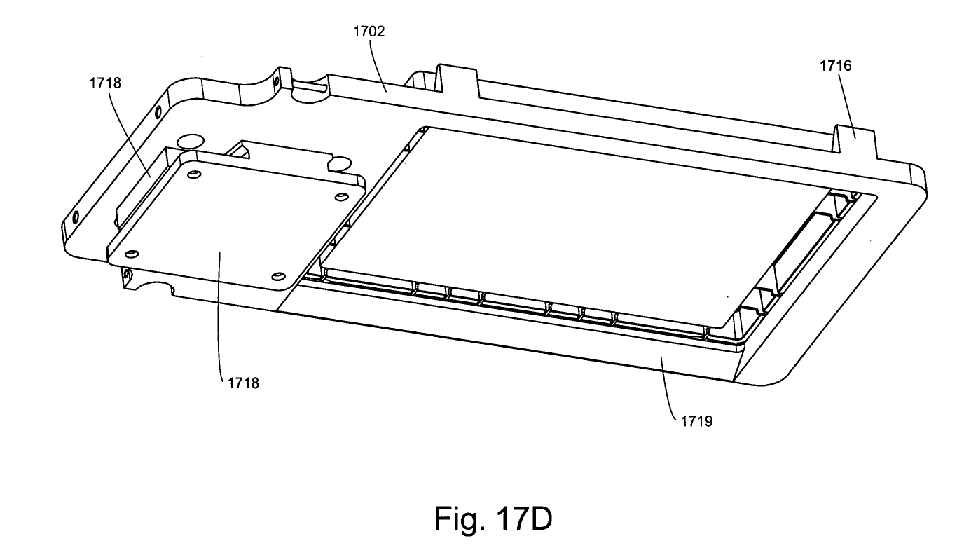

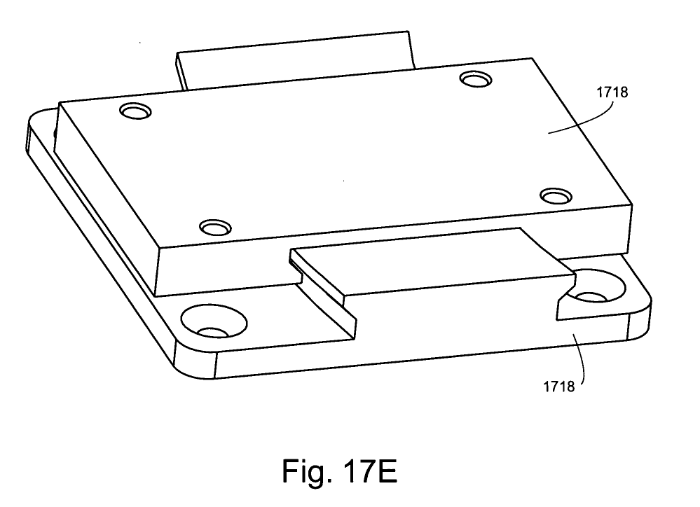

The container positioning devices of the present invention also include other embodiments. For example, FIG. 17A schematically shows container positioning device 1700 from a perspective view. As shown, container positioning device 1700 includes nests 1702, 1704, 1706, and 1708 in which multi-well containers can be placed to position the containers relative to the fluid transfer device. Nests 1702, 1704, 1706, and 1708 are typically precisely fabricated (e.g., machined, molded, etc.) such that sample plates fit tightly (i.e., substantially without room for lateral movement, etc.) into nests 1702, 1704, 1706, and 1708. Component fabrication is described further below. As shown, nests 1702, 1704, 1706, and 1708 each include multiple alignment members 1716 that include angled surfaces that are configured to direct multi-well containers into nests 1702, 1704, 1706, and 1708, respectively, when such containers are placed into those nests. Nests 1702 and 1704 are fabricated to rotate about the centers of plates positioned in those nests so that plate positions can be adjusted to align with the pin tool of the fluid transfer device. This eliminates the need to include a corresponding rotational adjustment in, e.g., the pin tool and/or fluid transfer device chassis. However, in some embodiments, these other rotational adjustments are also included for additional control over the alignment of the pin tool and plates.

FIG. 17B schematically shows positioning device 1700 of FIG. 17A from a partially exploded perspective view. As shown, nest 1702 and 1704 rotate about rotational coupling components 1718 (shown as a carriage and base that mate via a dovetail joint) that mate with or otherwise contact both the particular nest and top tier support structure component 1710 of positioning device 1700, which are typically disposed proximal to an end of the particular nest. Rotational coupling components 1718 are typically fabricated from stainless steel with a thin (e.g., 0.002 inches thick) brass, TEFLON™, or other shim inserted between the two pieces to provide a bearing surface. Other rotational couplings, which are generally known in the art, are also optionally utilized. The rotational positions of nests 1702 and 1704 are individually adjusted using set screws 1714 and 1712, respectively, or other functionally equivalent rotational adjustment features. Springs 1715 provide counteracting tension to set screws 1714 and 1712 to maintain the selected rotational position of nests 1702 and 1704. In addition, nest 1702 includes orifice or cutout 1720 so that when a container is positioned over the orifice 1720, the container can receive electromagnetic radiation from an electromagnetic source and/or the detector can receive electromagnetic radiation from the container through orifice (e.g., via an optical system, etc.). Additional details relating to the container positioning devices of the present invention are described in, e.g., International Publication No. WO 01/96880, entitled “AUTOMATED PRECISION OBJECT HOLDER,” filed Jun. 15, 2001 by Mainquist et al., and Attorney Docket No. 36-003700US, entitled “MULTI-WELL CONTAINER POSITIONING DEVICES AND RELATED SYSTEMS AND METHODS,” filed Aug. 4, 2003 by Evans, which are incorporated by reference in their entirety.

To further illustrate the invention, FIG. 17C schematically shows a partially transparent top view of a portion of nest 1702 of positioning device 1700. The relative orientation of rotational coupling components 1718 is shown. This is further depicted in FIG. 17D, which schematically shows nest 1702 from a bottom perspective view. As shown, edge 1719 includes an angled cut surface (e.g., at approximately 45°) to allow, e.g., electromagnetic radiation from an excitation laser or other electromagnetic radiation source to be incident on any selected well of a given multi-well container without being obstructed the nest structure. These angled edges are also typically included in other container stations having orifices as described herein. In addition, FIG. 17E schematically depicts a detailed perspective view of rotational coupling components 1718.

Nests 1706 and 1708 are optionally used to position additional sample plates. In some embodiments, at least one of nests 1706 and 1708 is used as a fluid transfer probe or pin tool blotting station to remove adherent fluid from the probe before or after a fluid transfer step is performed. In these embodiments, blotting paper (not shown) is placed in, e.g., nest 1706 and pin tool 120 is contacted with the paper such that adherent fluid is blotted, wicked, or otherwise removed from the pins of pin tool 120. Various types of blotting paper including, e.g., lint-free blotting paper, etc. are commercially available from many different suppliers, such as V&P Scientific, Inc. (San Diego, Calif.) or the like.

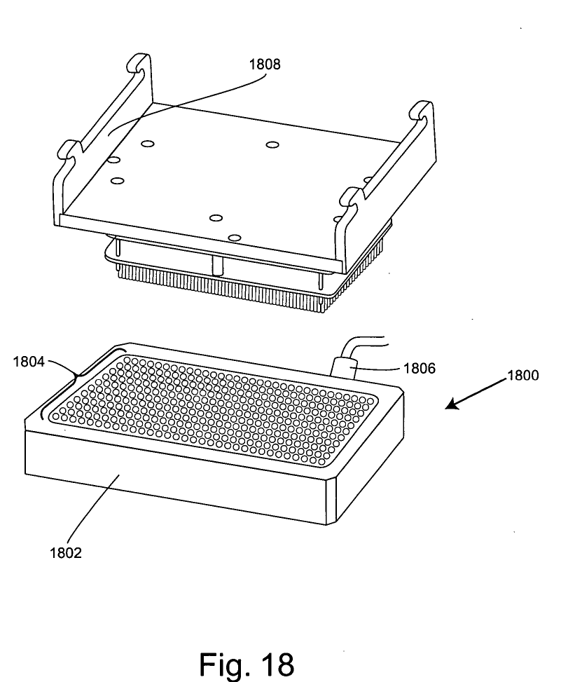

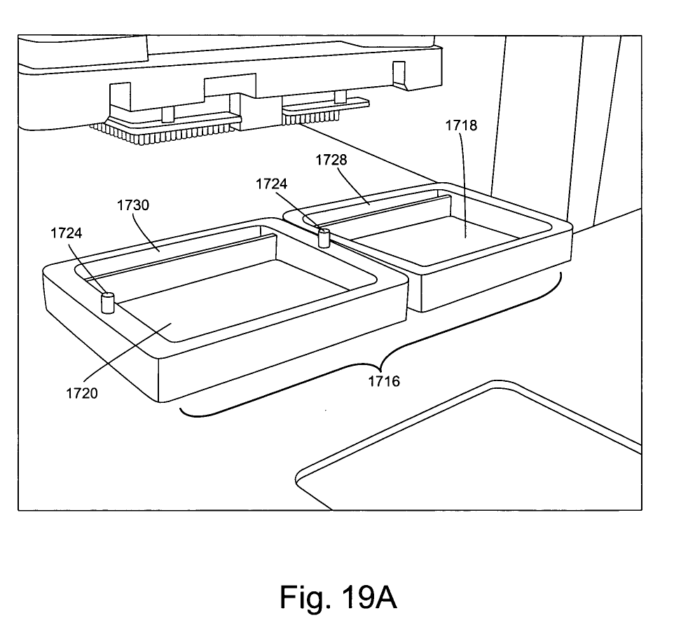

In certain embodiments, the system further includes a fluid transfer probe or pin tool vacuum drying station that removes adherent fluid from the pins under an applied vacuum when the pin tool is disposed proximal to the vacuum drying station. Optionally, such a vacuum drying station replaces, e.g., nest 1706 and/or nest 1708 or is positioned at another location that is either internal or external to the assaying system of the invention. An exemplary fluid transfer probe vacuum drying station is schematically depicted in FIG. 18. As shown, vacuum drying station 1800 includes vacuum drying station body structure 1802, which includes array of holes 1804 through which vacuum is applied to effect the removal of adherent fluid from the pins of pin tool 1808 when the pins are positioned proximal to array of holes 1804 by the fluid transfer device. In some embodiments, vacuum holes are arrayed to have a footprint that corresponds to the pins of the particular pin tool being utilized (e.g., a one-to-one correspondence). In other embodiments, a one-to-one correspondence between the number of vacuum holes and the number of pins is not present. For example, if there are fewer holes in the particular array than in the pin tool, then the applied vacuum is typically increased so that a given hole can remove adherent fluid from multiple pins. Vacuum is typically applied via a vacuum line operably connected to vacuum port 1806.

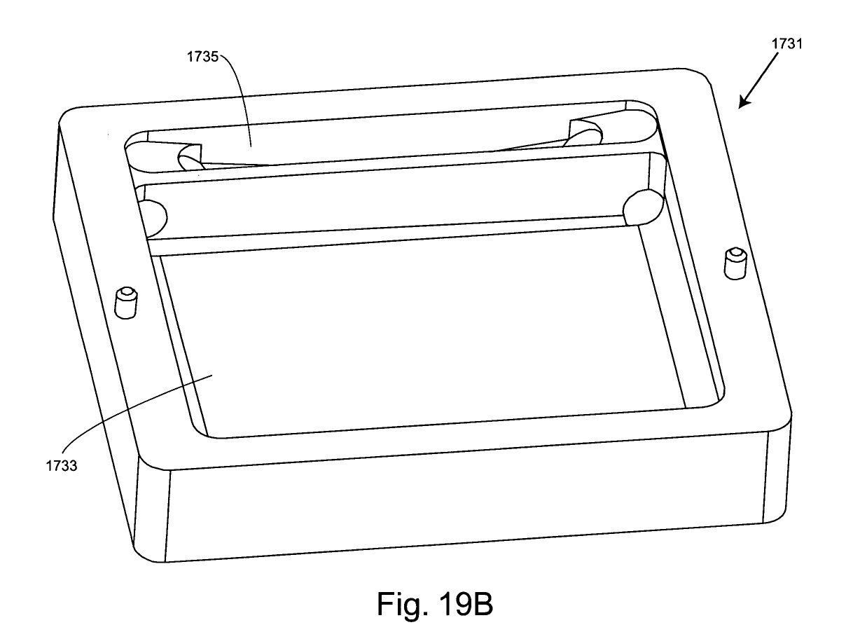

As additionally shown in FIG. 17A, container positioning device 1700 also includes fluid transfer probe washing station 1716, which includes wash reservoirs 1718 and 1720 (e.g., recirculation troughs or baths, etc.) disposed on bottom tier support structure component 1722 of container positioning device 1700. Wash reservoirs 1718 and 1720 are generally filled with a wash solvent such as dimethyl sulfoxide (DMSO), ethanol, methanol, water, or the like and are used to wash pin tool 120. For example, one washing or cleaning protocol includes filling wash reservoir 1720 with DMSO and filling wash reservoir 1718 with ethanol (or methanol). In this cleaning protocol, after compounds are transferred from a compound plate to an assay plate, the pins of pin tool 120 are first dipped into the DMSO bath, followed by being dipped into the ethanol (or methanol) bath. In embodiments that include the blotting stations described above, the pins are then typically contacted with the blotting paper to remove the wash solvent. As one alternative to this wash protocol, after compound transfer, the pins are blotted before being dipped into wash reservoirs 1720 and 1718, as described above. As also shown, fluid transfer probe washing station 1716 also includes overflow reservoir 1726 that fluidly communicates wash reservoir 1718 by reservoir divider 1728, which is disposed below the level of the openings to wash reservoir 1718 and overflow reservoir 1726. Overflow reservoir 1726 prevents wash solvent from overflowing from wash reservoir 1718, e.g., onto other components of the system. Although not within view in FIG. 17A, an overflow reservoir also fluidly communicates with wash reservoir 1720. This is illustrated in FIG. 19A, which schematically shows fluid transfer probe washing station 1716 from a perspective view. As shown, overflow reservoir 1730 fluidly communicates with wash reservoir 1720. To further illustrate another exemplary embodiment, FIG. 19B shows fluid transfer probe washing station 1731, which includes wash reservoir 1733 and overflow reservoir 1735.

Optionally, at least one of wash reservoirs 1718 and 1720 is used as a docking station for pin tool 120 when it is not attached to the chassis of the fluid transfer device. As shown in FIG. 17A, for example, wash reservoir 1720 includes first alignment features 1724 (e.g., pins, etc.)(one not within view) and a floating plate of pin tool 120 includes second alignment features (e.g., holes, etc.)(one not within view) that correspond to first alignment features 1724. For example, when the fluid transfer device dips pin tool 120 into wash reservoir 1720, first alignment features 1724 and the corresponding second alignment features mate with one another to align pin tool 120 relative to wash reservoir 1720 such that the fluid transfer device chassis can detach from pin tool 120. These alignment features also align pin tool 120 and wash reservoir 1720 when the pins are washed, e.g., according to a wash protocol described herein.

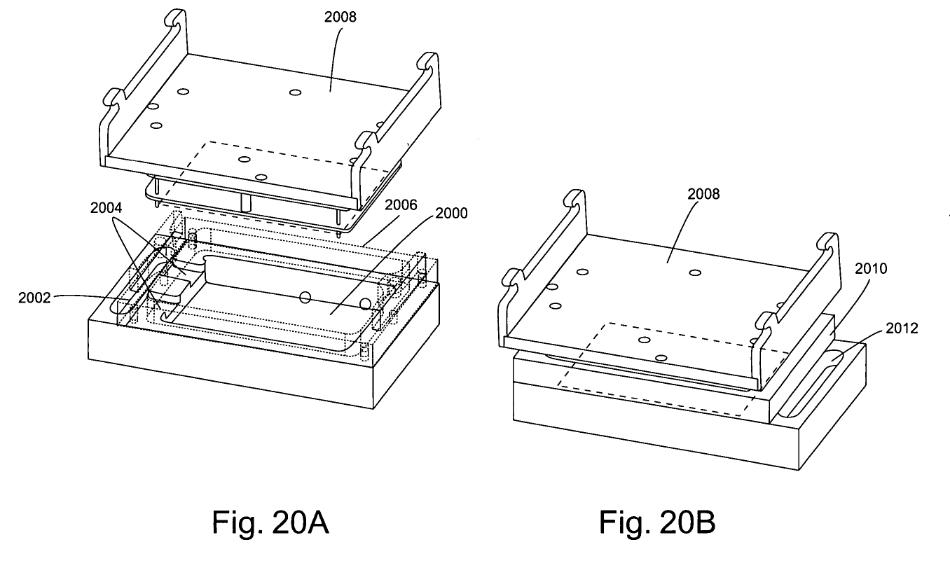

To illustrate another embodiment, FIG. 20A schematically shows wash reservoir 2000 from a perspective view. As shown, wash reservoir 2000 fluidly communicates with overflow reservoir 2002 via overflow channels 2004. FIG. 20A also shows a transparent perspective view of non-pressure-based fluid transfer probe mount 2006 disposed around wash reservoir 2000. Non-pressure-based fluid transfer probe mount 2006 is optionally utilized to mount or position non-pressure-based fluid transfer probe 2008 relative to wash reservoir 2000 when non-pressure-based fluid transfer probe 2008 is washed and/or when non-pressure-based fluid transfer probe 2008 is separated from a chassis of the fluid transfer device. In addition, FIG. 20B schematically shows non-pressure-based fluid transfer probe 2008 positioned or mounted on non-pressure-based fluid transfer probe mount 2010 from a perspective view. As shown, the wash reservoir (not within view) and overflow reservoir 2012 mirror the orientation of wash reservoir 2000 and non-pressure-based fluid transfer probe mount 2006 depicted in FIG. 20A.

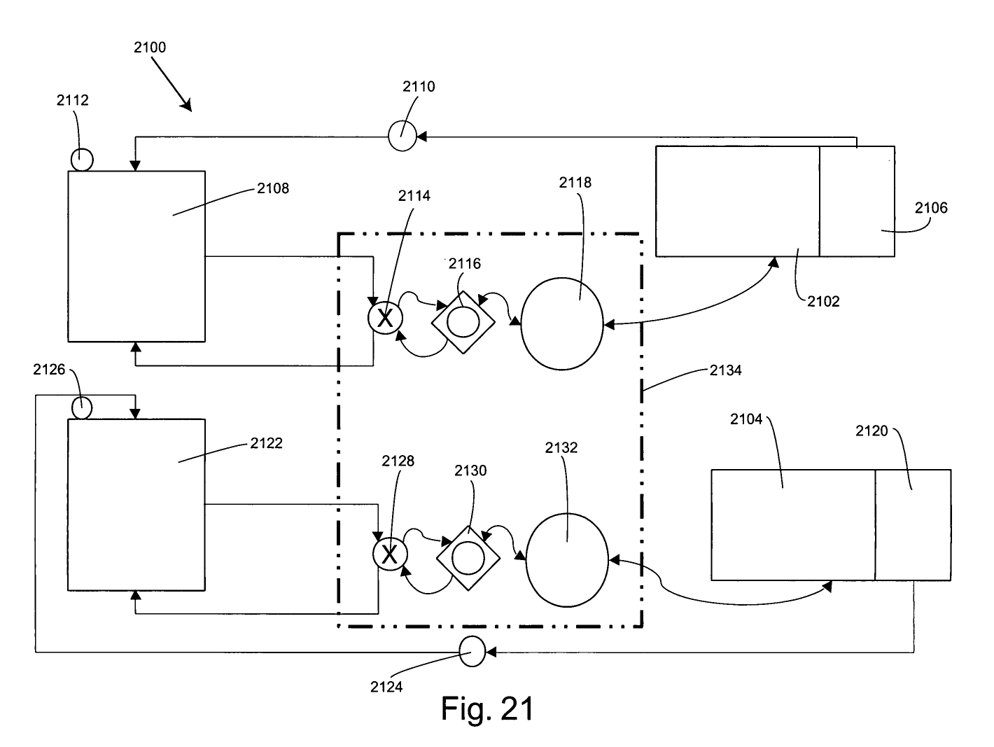

FIG. 21 is a block diagram showing representative fluid transfer probe washing station 2100. As shown, fluid transfer probe washing station 2100 includes two wash reservoirs, namely, wash reservoir 2102 and wash reservoir 2104. Wash reservoirs 2102 and 2104 typically contain different wash solvents (e.g., DMSO, ethanol, methanol, water, or the like). Wash reservoir 2102 fluidly communicates with overflow reservoir 2106, which fluidly communicates with waste reservoir 2108 via a fluid conduit. As shown, fluid sensor 2110 is disposed in sensory communication with the fluid conduit between wash reservoir 2102 and overflow reservoir 2106 to sense fluid disposed proximal to (e.g., leakage from, etc.) the fluid conduit. Fluid sensor 2112 is disposed in sensory communication with waste reservoir 2108 to sense fluid disposed proximal to and/or the fluid level in waste reservoir 2108. Fluid sensors utilized in fluid transfer probe washing station 2100 are optionally wet or dry sink fluid presence sensors. In addition, the fluid sensors of fluid transfer probe washing station 2100 are typically operably connected to one or more controllers, which receive data from the fluid sensors to monitor the presence of fluid in and/or proximal to fluid transfer probe washing station 2100. Controllers are described in greater detail below. Wash reservoir 2102 and waste reservoir 2108 also fluidly communicate with one another via valve 2114 (e.g., a three-way pinch valve or the like), fluid sensor 2116, and pump 2118 (e.g., a peristaltic pump, etc.). Pump 2118 effects fluid flow between wash reservoir 2102 and waste reservoir 2108.

As additionally shown in FIG. 21, wash reservoir 2104 fluidly communicates with overflow reservoir 2120, which fluidly communicates with waste reservoir 2122 via a fluid conduit. As also shown, fluid sensor 2124 is disposed in sensory communication with the fluid conduit between wash reservoir 2104 and overflow reservoir 2122 to sense fluid disposed proximal to (e.g., leakage from, etc.) the fluid conduit. Fluid sensor 2126 is disposed in sensory communication with waste reservoir 2108 to sense fluid disposed proximal to and/or the fluid level in waste reservoir 2122. Wash reservoir 2104 and waste reservoir 2122 also fluidly communicate with one another via valve 2128 (e.g., a three-way pinch valve or the like), fluid sensor 2130, and pump 2132 (e.g., a peristaltic pump, etc.). Pump 2132 effects fluid flow between wash reservoir 2104 and waste reservoir 2122. Valves 2114 and 2128, fluid sensors 2116 and 2130, and pumps 2118 and 2132 are typically housed in electronics box 2134. In addition, one or more controllers (e.g., pump and valve controllers, etc.) and a power supply are also optionally housed in electronics box 2134.

D. Electromagnetic Radiation Sources, Optical Systems, and Detectors

The sample assaying systems of the invention are configured to detect and quantify absorbance, transmission, and/or emission of light, and/or changes in those properties in samples that are typically arrayed in the wells of a multi-well plate, or arrayed in dot blots supported on membranes, treated glass, or other support materials. The systems of the invention can also be used to detect and quantify these properties in irregularly distributed samples. In addition to other system components described herein, the assaying systems of the invention also generally include illumination or electromagnetic radiation sources, optical systems, and detectors. Because the systems and methods of the invention are flexible and allow essentially any chemistry to be assayed, they can be used for all phases of assay development, including prototyping and mass screening.

In preferred embodiments, the assaying systems of the invention are configured for area imaging, but can also be configured for other formats including as a scanning imager or as a nonimaging counting system. An area imaging system typically places an entire multi-well container or other specimen onto the detector plane at one time. Accordingly, there is typically no need to move photomultiplier tubes (PMTs), to scan a laser, or the like, because the detector images the entire container onto many small detector elements (e.g., charge-coupled devices (CCDs), etc.) in parallel. This parallel acquisition phase is typically followed by a serial process of reading out the entire image from the detector. Scanning imagers typically pass a laser or other light beam over the specimen, to excite fluorescence, reflectance, or the like in a point-by-point or line-by-line fashion. In certain cases, confocal-optics are used to minimize out of focus fluorescence. The image is constructed over time by accumulating the points or lines in series. Nonimaging counting systems typically use PMTs or light sensing diodes to detect alterations in the transmission or emission of light, e.g., within wells of a multi-well container. These systems then typically integrate the light output from each well into a single data point.

A wide variety of illumination or electromagnetic sources and optical systems can be adapted for use in the systems of the present invention. Accordingly, no attempt is made herein to describe all of the possible variations that can be utilized in the systems of the invention and which will be apparent to one skilled in the art. Exemplary electromagnetic radiation sources that are optionally utilized in the systems of the invention include, e.g., lasers, laser diodes, electroluminescence devices, light-emitting diodes, incandescent lamps, arc lamps, flash lamps, fluorescent lamps, and the like. One preferred type of laser used in the assaying systems of the invention are argon-ion lasers. Exemplary optical systems that conduct electromagnetic radiation from electromagnetic radiation sources to sample containers and/or from sample containers to detectors typically include one or more lenses and/or mirrors to focus and/or direct the electromagnetic radiation as desired. Many optical systems also include fiber optic bundles, optical couplers, filters (e.g., filter wheels, etc.), and the like.

Suitable signal detectors that are optionally utilized in these systems detect, e.g., emission, luminescence, transmission, fluorescence, phosphorescence, absorbance, or the like. In preferred embodiments, the detector monitors a plurality of optical signals, which correspond in position to “real time” results. Example detectors or sensors include PMTs, CCDs, intensified CCDs, photodiodes, avalanche photodiodes, optical sensors, scanning detectors, or the like. Each of these as well as other types of sensors is optionally readily incorporated into the systems described herein. The detector optionally moves relative to multi-well plates or other assay components, or alternatively, multi-well plates or other assay components move relative to the detector. In certain embodiments, for example, detection components are coupled to translation components that move the detection components relative to multi-well plates positioned on container positioning devices of the systems described herein. Optionally, the systems of the present invention include multiple detectors. In these systems, such detectors are typically placed either in or adjacent to, e.g., a multi-well plate or other vessel, such that the detector is in sensory communication with the multi-well plate or other vessel (i.e., the detector is capable of detecting the property of the plate or vessel or portion thereof, the contents of a portion of the plate or vessel, or the like, for which that detector is intended). In preferred embodiments, detectors are configured to detect electromagnetic radiation originating in the wells of a multi-well container.

The detector optionally includes or is operably linked to a computer, e.g., which has system software for converting detector signal information into assay result information or the like. For example, detectors optionally exist as separate units, or are integrated with controllers into a single instrument. Integration of these functions into a single unit facilitates connection of these instruments with the computer, by permitting the use of a few or even a single communication port for transmitting information between system components. Computers and controllers are described further below. Detection components that are optionally included in the systems of the invention are described further in, e.g., Skoog et al., Principles of Instrumental Analysis, 5th Ed., Harcourt Brace College Publishers (1998) and Currell, Analytical Instrumentation: Performance Characteristics and Quality, John Wiley & Sons, Inc. (2000), which are incorporated by reference in their entirety.

Additional details relating to electromagnetic radiation sources, optical systems, detectors, and other aspects of the present invention which can be utilized or adapted for use in the systems described herein are provided in, e.g., U.S. Pat. No. 6,316,774, entitled “OPTICAL SYSTEM FOR A SCANNING FLUOROMETER,” which issued Nov. 13, 2001 to Giebeler et al., U.S. Pat. No. 5,112,134, entitled “SINGLE SOURCE MULTI-SITE PHOTOMETRIC MEASUREMENT SYSTEM,” which issued May 12, 1992 to Chow et al., U.S. Pat. No. 5,766,875, entitled “METABOLIC MONITORING OF CELLS IN A MICROPLATE READER,” which issued Jun. 16, 1998 to Hafeman et al., U.S. Pat. No. 6,469,311, entitled “DETECTION DEVICE FOR LIGHT TRANSMITTED FROM A SENSED VOLUME,” which issued Oct. 22, 2002 to Modlin et al., U.S. Pat. No. 6,151,111, entitled “PHOTOMETRIC DEVICE,” which issued Nov. 21, 2000 to Wechsler et al., U.S. Pat. No. 6,498,690, entitled “DIGITAL IMAGING SYSTEM FOR ASSAYS IN WELL PLATES, GELS AND BLOTS,” which issued Dec. 24, 2002 to Ramm et al., and U.S. Pat. No. 6,313,471, entitled “SCANNING FLUOROMETER,” which issued Nov. 6, 2001 to Giebeler et al.

E. Contollers

The sample assaying systems of the invention also typically include controllers that are operably connected to one or more components (e.g., electromagnetic radiation sources, fluid transfer devices, detectors, valves, pumps, fluid sensors, translocation components, robotic handlers, container positioning devices, etc.) of the systems to control operation of the components. More specifically, controllers are generally included either as separate or integral system components that are utilized, e.g., to regulate the intensity and/or wavelength of electromagnetic radiation emitted from the electromagnetic radiation source, the movement of fluid transfer devices, the detection of electromagnetic radiation received from sample containers by the detector, etc. Controllers and/or other system components is/are optionally coupled to an appropriately programmed processor, computer, digital device, or other logic device or information appliance (e.g., including an analog to digital or digital to analog converter as needed), which functions to instruct the operation of these instruments in accordance with preprogrammed or user input instructions, receive data and information from these instruments, and interpret, manipulate and report this information to the user.

Any controller or computer optionally includes a monitor which is often a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard or mouse optionally provide for input from a user. An exemplary computer is schematically illustrated in FIG. 1.

The computer typically includes appropriate software for receiving user instructions, either in the form of user input into a set of parameter fields, e.g., in a GUI, or in the form of preprogrammed instructions, e.g., preprogrammed for a variety of different specific operations. The software then converts these instructions to appropriate language for instructing the operation of one or more controllers to carry out the desired operation, e.g., varying or selecting the rate or mode of movement of various system components, directing translation of robotic gripping devices and fluid transfer devices, or the like. The computer then receives the data from, e.g., sensors/detectors included within the system, and interprets the data, either provides it in a user understood format, or uses that data to initiate further controller instructions, in accordance with the programming, e.g., such as in monitoring detectable signal intensity, multi-well container positioning, or the like.

More specifically, the software utilized to control the operation of the systems of the invention typically includes logic instruction instructions that direct, e.g., the fluid transfer device to attach and/or detach a pin tool to or from the chassis, the fluid transfer device to transfer fluid between containers with a pin tool, the pushers of the container positioning device to push the containers into contact with the alignment members when the containers are positioned in a container station, the fluid transfer device to position the fluid transfer probe in the wash reservoir to effect washing of a fluid transfer probe, the movement of container moving component, a robotic device to translocate containers, the fluid transfer device to blot away the fluid that adheres to the non-pressure-based fluid transfer probe (e.g., at a blotting station, if included in the system), the fluid transfer device to move the fluid transfer probe proximal to a fluid transfer probe vacuum drying station and the fluid transfer probe vacuum drying station to dry the fluid transfer probe, and/or the like.

The computer can be, e.g., a PC (Intel x86 or Pentium chip-compatible DOS™, OS2™, WINDOWS™, WINDOWS NT™, WINDOWS95™, WINDOWS98™, WINDOWS2000™, WINDOWS XP™, LINUX-based machine, a MACINTOSH™, Power PC, or a UNIX-based (e.g., SUN™ work station) machine) or other common commercially available computer which is known to one of skill. Standard desktop applications such as word processing software (e.g., Microsoft Word™ or Corel WordPerfect™) and database software (e.g., spreadsheet software such as Microsoft Excel™, Corel Quattro PrO™, or database programs such as Microsoft Access™ or Paradox™) can be adapted to the present invention. Software for performing, e.g., fluid transfer to selected wells of a multi-well plate, assay detection, and data deconvolution is optionally constructed by one of skill using a standard programming language such as Visual basic, Fortran, Basic, Java, or the like.

F. Additional System Components

The systems of the invention optionally further include various container incubation components and/or container storage components. In some embodiments, for example, systems include incubation components that are structured to incubate or regulate temperatures within multi-well plates. To illustrate, many cell-based or other types of assays include incubation steps and can be performed using these systems. Additional details regarding incubation devices that are optionally adapted for use with the systems of the present invention are described in, e.g., International Publication No. WO 03/008103, entitled “HIGH THROUGHPUT INCUBATION DEVICES,” filed Jul. 18, 2002 by Weselak et al., which is incorporated by reference in its entirety. In certain embodiments, the sample assaying systems of the invention include multi-well plate storage components that are structured to store one or more multi-well plates. Such storage components typically include multi-well plate hotels or carousels that are known in the art and readily available from various commercial suppliers, such as Beckman Coulter, Inc. (Fullerton, Calif.). For example, in one embodiment, a system of the invention includes a stand-alone station in which a user loads a number of multi-well plates to be assayed into one or more storage components of the system for automated processing of the plates. In these embodiments, the systems of the invention also typically include one or more robotic gripper devices that move plates, e.g., between incubation or storage components and container positioning devices. Robotic grippers that are suitable for use in the systems of the invention are described further below or otherwise known in the art. For example, a TECAN® robot, which is commercially available from Clontech (Palo Alto, Calif.), is optionally adapted for use in the systems described herein. An exemplary container storage component is schematically shown in FIG. 1.

The systems of the invention optionally also include at least one robotic gripping component that is structured to grip and translocate multi-well plates between components of the sample assaying systems and/or between the sample assaying systems and other locations (e.g., other work stations, etc.). In certain embodiments, for example, systems further include gripping components that move multi-well plates between container positioning components, incubation components, etc. A variety of available robotic elements (robotic arms, movable platforms, etc.) can be used or modified for use with these systems, which robotic elements are typically operably connected to controllers that control their movement and other functions. Exemplary robotic gripping devices that are optionally adapted for use in the systems of the invention are described further in, e.g., International Publication No. WO 02/068157, entitled “GRIPPING MECHANISMS, APPARATUS, AND METHODS,” filed Feb. 26, 2002 by Downs et al., which is incorporated by reference in its entirety.

G. Assaying System Component Manufacture

System components (e.g., container positioning device components, fluid transfer device components, washing station components, etc.) are optionally formed by various fabrication techniques or combinations of such techniques including, e.g., machining, stamping, engraving, injection molding, cast molding, embossing, extrusion, etching (e.g., electrochemical etching, etc.), or other techniques. These and other suitable fabrication techniques are generally known in the art and described in, e.g., Altintas, Manufacturing Automation: Metal Cutting Mechanics, Machine Tool Vibrations, and CNC Design, Cambridge University Press (2000), Molinari et al. (Eds.), Metal Cutting and High Speed Machining, Kluwer Academic Publishers (2002), Stephenson et al., Metal Cutting Theory and Practice, Marcel Dekker (1997), Rosato, Injection Molding Handbook, 3^{rd} Ed., Kluwer Academic Publishers (2000), Fundamentals of Injection Molding, W. J. T. Associates (2000), Whelan, Injection Molding of Thermoplastics Materials, Vol. 2, Chapman & Hall (1991), Fisher, Extrusion of Plastics, Halsted Press (1976), and Chung, Extrusion of Polymers: Theory and Practice, Hanser-Gardner Publications (2000). In certain embodiments, following fabrication system components are optionally further processed, e.g., by coating surfaces with a hydrophilic coating, a hydrophobic coating (e.g., a Xylan 1010DF/870 Black coating available from Whitford Corporation (West Chester, Pa.), etc.), or the like, e.g., to prevent interactions between component surfaces and reagents, samples, or the like.

System component fabrication materials are generally selected according to properties, such as reaction inertness, durability, expense, or the like. In preferred embodiments, components are fabricated from various metallic materials, such as stainless steel, anodized aluminum, or the like. Optionally, system components are fabricated from polymeric materials such as, polytetrafluoroethylene (TEFLON™), polypropylene, polystyrene, polysulfone, polyethylene, polymethylpentene, polydimethylsiloxane (PDMS), polycarbonate, polyvinylchloride (PVC), polymethylmethacrylate (PMMA), or the like. Polymeric parts are typically economical to fabricate, which affords disposability. Component parts are also optionally fabricated from other materials including, e.g., glass, silicon, or the like.

H. Methods of Assaying Samples

The present invention also relates to methods of assaying fluidic samples using the systems described herein. Essentially any biochemical or cellular assay can be adapted for performance in the systems of the invention. Exemplary assays optionally performed in the systems described herein include, e.g., intracellular calcium flux assays, membrane potential assays, nucleic acid hybridization assays among many others that are known in the art. The methods typically include positioning at least a first multi-well container in the sample assaying region, and transferring fluidic samples (e.g., drug candidates and target molecules, samples comprising cells, combinatorial library members, labeled molecules, etc.) from at least a second multi-well container to the first container with a non-pressure-based fluidic transfer probe (e.g., a pin tool, etc.) of the fluid transfer device of the system. The positioning step generally includes placing the first container in the sample assaying region with a robotic device. The method also includes detecting sample electromagnetic radiation received from the first container with the detector when the first container receives source electromagnetic radiation from the electromagnetic radiation source. The detecting step typically includes imaging the sample electromagnetic radiation received from the first container over time.

The transferring step typically includes transferring multiple fluidic samples from the second container and/or at least a third container to the first container. Optionally, the method also includes washing, blotting, and/or drying the non-pressure-based fluidic transfer probe after at least one of the fluidic samples is transferred to the first fluid container. As mentioned, the non-pressure-based fluidic transfer probe typically includes a pin tool having multiple pins and the transferring step includes substantially simultaneously transferring multiple samples from the second container to the first container. In these embodiments, the fluid transfer device generally includes at least one chassis and the pin tool includes a support structure having at least one attachment feature that removably attaches to the chassis, and the method typically further includes attaching the pin tool to the chassis before the transferring step or detaching the pin tool from the chassis after the transferring step. Optionally, the method further includes rotating the pin tool and/or the first container relative to one another before or after transferring the multiple samples to the first sample container, e.g., to align the pin tool and the first container with one another.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

1. A sample assaying system, comprising:
at least one electromagnetic radiation source;
at least one sample assaying region configured to receive source electromagnetic radiation from the electromagnetic radiation source;
at least one fluid transfer device comprising at least one non-pressure-based fluid transfer probe, which fluid transfer device is configured to transfer fluid in at least one selected region of the sample assaying system; and,
at least one detector configured to detect sample electromagnetic radiation received from the sample assaying region.

2. The sample assaying system of claim 1, wherein the electromagnetic radiation source is selected from the group consisting of: a laser, a laser diode, an electroluminescence device, a light-emitting diode, an incandescent lamp, an arc lamp, a flash lamp, and a fluorescent lamp.

3. The sample assaying system of claim 1, wherein the sample assaying region comprises at least one thermal modulation nest.

4. The sample assaying system of claim 1, wherein the selected region comprises the sample assaying region.

5. The sample assaying system of claim 1, wherein the non-pressure-based fluid transfer probe is removably attached to the fluid transfer device.

6. The sample assaying system of claim 1, wherein the non-pressure-based fluid transfer probe comprises a pin.

7. The sample assaying system of claim 1, wherein the detector is selected from the group consisting of: a charge-coupled device, an intensified charge-coupled device, a photomultiplier tube, a photodiode, and an avalanche photodiode.

8. The sample assaying system of claim 1, further comprising at least one controller operably connected at least to the electromagnetic radiation source, the fluid transfer device, and the detector, which controller comprises at least one logic device having one or more logic instructions that direct operation of the electromagnetic radiation source, the fluid transfer device, and the detector.

9. The sample assaying system of claim 1, further comprising at least one container storage component that is structured to store one or more containers.

10. The sample assaying system of claim 1, further comprising at least one container incubation component that is structured to incubate one or more containers.

11. The sample assaying system of claim 1, further comprising a container moving component that is structured to move one or more containers at least relative to the fluid transfer device.

12. The sample assaying system of claim 11, further comprising at least one controller operably connected to the container moving component, which controller comprises at least one logic device having one or more logic instructions that direct movement of the container moving component.

13. The sample assaying system of claim 1, wherein the non-pressure-based fluid transfer probe comprises a pin tool that comprises 6, 12, 24, 48, 96, 192, 384, 768, 1536, 3456, 9600, or more pins.

14. The sample assaying system of claim 13, wherein the fluid transfer device comprises at least one chassis and the pin tool comprises a support structure having at least one attachment feature that removably attaches to the chassis.

15. The sample assaying system of claim 14, wherein the attachment feature comprises a hook.

16. The sample assaying system of claim 14, further comprising at least one controller operably connected to the fluid transfer device, which controller comprises at least one logic device having one or more logic instructions that direct the fluid transfer device to attach and/or detach the pin tool to or from the chassis, and the fluid transfer device to transfer fluid between containers with the pin tool.

17. The sample assaying system of claim 14, wherein the pin tool comprises a pin tool head having a rotational adjustment feature such that the pin tool head is capable of rotating relative to the support structure.

18. The sample assaying system of claim 17, wherein the rotational adjustment feature comprises a screw.

19. The sample assaying system of claim 17, wherein the pin tool head is removably attached to the support structure by one or more attachment components.

20. The sample assaying system of claim 19, wherein the attachment components comprise set screws and/or spring ball sockets.

21. The sample assaying system of claim 1, wherein the sample assaying region comprises at least one container positioning device, which container positioning device comprises at least one container station that is structured to position at least one container or other support relative to the fluid transfer device.

22. The sample assaying system of claim 21, wherein the container station comprises a heating element to regulate temperature in the container or on the other support.

23. The sample assaying system of claim 21, wherein the container station is structured to position at least one multi-well container that comprises 6, 12, 24, 48, 96, 192, 384, 768, 1536, 3456, 9600, or more wells.

24. The sample assaying system of claim 21, wherein the container station comprises a nest.

25. The sample assaying system of claim 21, wherein the container station is structured to rotate relative to the fluid transfer device.

26. The sample assaying system of claim 21, wherein the container station comprises at least one orifice disposed through the container positioning device such that when one or more containers are positioned in the container station, the containers receive the source electromagnetic radiation from the electromagnetic radiation source through the orifice and/or the detector receives the sample electromagnetic radiation from the containers through the orifice.

27. The sample assaying system of claim 21, wherein the container positioning device comprises multiple container stations.

28. The sample assaying system of claim 27, wherein at least two of the container stations are tiered.

29. The sample assaying system of claim 28, further comprising at least one robotic handler capable of handling a first container positioned in one tiered container station without contacting a second container positioned in another tiered container station.

30. The sample assaying system of claim 21, wherein the container positioning device comprises one or more alignment members that are positioned to contact one or more surfaces of one or more containers when the containers are positioned in the container station such that the containers align with the fluid transfer device.

31. The sample assaying system of claim 30, wherein alignment member receiving areas disposed in bottom surfaces of the containers comprise the surfaces that contact the alignment members.

32. The sample assaying system of claim 30, further comprising one or more pushers that are capable of pushing the containers into contact with the alignment members when the containers are positioned in the container station.

33. The sample assaying system of claim 32, further comprising at least one controller operably connected to the container positioning device, which controller comprises at least one logic device having one or more logic instructions that direct the pushers to push the containers into contact with the alignment members when the containers are positioned in the container station.

34. The sample assaying system of claim 1, further comprising at least one fluid transfer probe washing station that comprises at least one wash reservoir structured to wash the non-pressure-based fluid transfer probe.

35. The sample assaying system of claim 34, wherein the wash reservoir comprises at least one overflow reservoir in fluid communication with the wash reservoir to receive fluid overflow from the wash reservoir.

36. The sample assaying system of claim 34, wherein the wash reservoir comprises at least one mount to position the non-pressure-based fluid transfer probe relative to the wash reservoir when the non-pressure-based fluid transfer probe is washed and/or when the non-pressure-based fluid transfer probe is separated from a chassis of the fluid transfer device.

37. The sample assaying system of claim 34, wherein the fluid transfer probe washing station comprises at least a first alignment feature and the non-pressure-based fluid transfer probe comprises at least a second alignment feature, which alignment features are capable of mating with one another to align the non-pressure-based fluid transfer probe relative to the fluid transfer probe washing station.

38. The sample assaying system of claim 34, further comprising at least one controller operably connected to the fluid transfer probe washing station, which controller directs operation of the fluid transfer probe washing station.

39. The sample assaying system of claim 34, further comprising at least one fluid sensor in sensory communication with at least one component of the fluid transfer probe washing station to sense fluid disposed proximal to the component.

40. The sample assaying system of claim 34, further comprising at least one waste reservoir in fluid communication with the wash reservoir by at least one fluid conduit.

41. The sample assaying system of claim 40, further comprising at least one pump operably connected to the fluid conduit to effect fluid flow through the conduit.

42. The sample assaying system of claim 40, further comprising at least one valve operably connected to the fluid conduit to regulate fluid flow through the fluid conduit.

43. The sample assaying system of claim 1, further comprising at least one robotic device configured to translocate containers at least between selected regions of the sample assaying system.

44. The sample assaying system of claim 43, further comprising at least one controller operably connected to the robotic device, which controller comprises at least one logic device having one or more logic instructions that direct the robotic device to translocate the containers.

45. The sample assaying system of claim 1, further comprising at least one fluid transfer probe blotting station structured to blot away fluid that adheres to the non-pressure-based fluid transfer probe.

46. The sample assaying system of claim 45, further comprising at least one controller operably connected to the fluid transfer device, which controller comprises at least one logic device having one or more logic instructions that direct the fluid transfer device to blot away the fluid that adheres to the non-pressure-based fluid transfer probe.

47. The sample assaying system of claim 1, further comprising at least one fluid transfer probe vacuum drying station structured to dry the non-pressure-based fluid transfer probe.

48. The sample assaying system of claim 47, further comprising at least one controller operably connected to the fluid transfer device and the fluid transfer probe vacuum drying station, which controller comprises at least one logic device having one or more logic instructions that direct the fluid transfer device to move the fluid transfer probe proximal to the fluid transfer probe vacuum drying station and the fluid transfer probe vacuum drying station to dry the fluid transfer probe.

49. A sample assaying system, comprising:
at least one electromagnetic radiation source;
at least one container positioning device comprising at least one container station that is structured to position at least one multi-well container, wherein the container station comprises at least one orifice disposed through the container positioning device such that when one or more multi-well containers are positioned in the container station at least one selected well disposed in the multi-well containers receives source electromagnetic radiation from the electromagnetic radiation source through the orifice;
at least one fluid transfer device comprising at least one chassis and at least one pin tool that removably attaches to the chassis, which pin tool is structured to transfer fluid to and/or from selected wells disposed in the multi-well container when the multi-well container is positioned in the container station; and,
at least one image detector configured to detect sample electromagnetic radiation received from the selected well disposed in the multi-well container through the orifice when the multi-well container is positioned in the container station.

50. A pin tool comprising a pin tool head having at least one pin attached to the pin tool head and a support structure having at least one attachment feature capable of removably attaching to a chassis of a fluid transfer device, wherein the pin tool head is removably attached to the support structure and the pin tool head comprises a rotational adjustment feature such that the pin tool head is capable of rotating relative to the support structure.

51. The pin tool of claim 50, wherein the attachment feature comprises a hook.

52. The pin tool of claim 50, wherein the pin tool head comprises 6, 12, 24, 48, 96, 192, 384, 768, 1536, 3456, 9600, or more pins.

53. The pin tool of claim 50, wherein the rotational adjustment feature comprises a screw.

54. The pin tool of claim 50, wherein the pin tool head is removably attached to the support structure by one or more attachment components.

55. The pin tool of claim 54, wherein the attachment components comprise set screws and/or spring ball sockets.

56. A method of assaying fluidic samples, the method comprising:
providing an assaying system that comprises:
at least one electromagnetic radiation source;
at least one sample assaying region configured to receive source electromagnetic radiation from the electromagnetic radiation source;
at least one fluid transfer device comprising at least one non-pressure-based fluid transfer probe, which fluid transfer device is configured to transfer fluid in at least one selected region of the sample assaying system; and
at least one detector;
positioning at least a first container in the sample assaying region;
transferring at least one fluidic sample from at least a second container to the first container with the non-pressure-based fluid transfer probe; and,
detecting sample electromagnetic radiation received from the first container with the detector when the first container receives source electromagnetic radiation from the electromagnetic radiation source, thereby performing the assay.

57. The method of claim 56, wherein the electromagnetic radiation source is selected from the group consisting of: a laser, a laser diode, an electroluminescence device, a light-emitting diode, an incandescent lamp, an arc lamp, a flash lamp, and a fluorescent lamp.

58. The method of claim 56, wherein the detector is selected from the group consisting of: a charge-coupled device, an intensified charge-coupled device, a photomultiplier tube, a photodiode, and an avalanche photodiode.

59. The method of claim 56, wherein the positioning step comprises placing the first container in the sample assaying region with a robotic device.

60. The method of claim 56, wherein the detecting step comprises imaging the sample electromagnetic radiation received from the first container over time.

61. The method of claim 56, wherein the transferring step comprises transferring multiple fluidic samples from the second container and/or at least a third container to the first container.

62. The method of claim 61, further comprising washing, blotting, and/or drying the non-pressure-based fluidic transfer probe after at least one of the fluidic samples is transferred to the first fluid container.

63. The method of claim 56, wherein the non-pressure-based fluidic transfer probe comprises a pin tool having multiple pins and the transferring step comprises substantially simultaneously transferring multiple samples from the second container to the first container.

64. The method of claim 63, wherein the fluid transfer device comprises at least one chassis and the pin tool comprises a support structure having at least one attachment feature that removably attaches to the chassis, and wherein the method further comprises attaching the pin tool to the chassis before the transferring step or detaching the pin tool from the chassis after the transferring step.

65. The method of claim 63, further comprising rotating the pin tool and/or the first container relative to one another before or after transferring the multiple samples to the first sample container.
